SEQUENCE LISTING (25 sequences)

SEQ ID NO: 1-2 - skipped (2 entries)

SEQ ID NO: 3 - protein, 8 aa, synthetic construct
  REGION 1..8: note = Synthetic construct
GVKVSTKS

SEQ ID NO: 4 - protein, 8 aa, synthetic construct
  REGION 1..8: note = Synthetic construct
GFKVTSQA

SEQ ID NO: 5 - protein, 8 aa, synthetic construct
  REGION 1..8: note = Synthetic construct.
  VAR_SEQ 2..5: note = X is any amino acid
IXXXXGST

SEQ ID NO: 6 - protein, 8 aa, synthetic construct
  REGION 1..8: note = Synthetic construct.
  VAR_SEQ 2..5: note = X is Ala, Arg, Lys, Asn, Gly, or Asp
IXXXXGST

SEQ ID NO: 7 - protein, 8 aa, synthetic construct
  REGION 1..8: note = Synthetic construct
IARNDGST

SEQ ID NO: 8 - protein, 8 aa, synthetic construct
  REGION 1..8: note = Synthetic Construct
IRKGNGST

SEQ ID NO: 9 - protein, 17 aa, synthetic construct
  REGION 1..17: note = Synthetic Construct
  VAR_SEQ 2: note = X is any amino acid
  VAR_SEQ 3: note = X is absent or is any amino acid
  VAR_SEQ 5..8: note = X is any amino acid
  VAR_SEQ 10..11: note = X is absent or is any amino acid
  VAR_SEQ 13..14: note = X is absent or is any amino acid
  VAR_SEQ 15..16: note = X is any amino acid
AXXKXXXXSXXQXXXXS

SEQ ID NO: 10 - protein, 17 aa, synthetic construct
  REGION 1..17: note = Synthetic construct
  VAR_SEQ 2: note = X is Arg, Thr, Lys, Ala, Asn, Tyr, Gly, Ile, or Leu
  VAR_SEQ 3: note = X is absent or Met, Lys, Arg, Pro, or His
  VAR_SEQ 5..8: note = X is Arg, Thr, Lys, Ala, Asn, Tyr, Gly, Ile, or Leu
  VAR_SEQ 10..11: note = X is absent or Met, Lys, Arg, Pro, or His
  VAR_SEQ 13..14: note = X is absent or Met, Lys, Arg, Pro, or His
  VAR_SEQ 15..16: note = X is Arg, Thr, Lys, Ala, Asn, Tyr, Gly, Ile, or Leu
AXXKXXXXSXXQXXXXS

SEQ ID NO: 11 - protein, 12 aa, synthetic construct
  REGION 1..12: note = Synthetic construct
  VAR_SEQ 2: note = X is any amino acid
  VAR_SEQ 4..7: note = X is any amino acid
  VAR_SEQ 10..11: note = X is any amino acid
AXKXXXXSQXXS

SEQ ID NO: 12 - protein, 12 aa, synthetic construct
  REGION 1..12: note = Synthetic construct.
  VAR_SEQ 2: note = X is Arg, Thr, Lys, Ala, Asn, Tyr, Gly, Ile or Leu
  VAR_SEQ 4..7: note = X is Arg, Thr, Lys, Ala, Asn, Tyr, Gly, Ile or Leu
  VAR_SEQ 10..11: note = X is Arg, Thr, Lys, Ala, Asn, Tyr, Gly, Ile or Leu
AXKXXXXSQXXS

SEQ ID NO: 13 - protein, 12 aa, synthetic construct
  REGION 1..12: note = Synthetic construct
  VAR_SEQ 2: note = X is Thr, Ala, Tyr, Ile, Lys, Leu, or Gly
  VAR_SEQ 4..7: note = X is Thr, Ala, Tyr, Ile, Lys, Leu, or Gly
  VAR_SEQ 10..11: note = X is Thr, Ala, Tyr, Ile, Lys, Leu, or Gly
AXKXXXXSQXXS

SEQ ID NO: 14 - protein, 17 aa, synthetic construct
  REGION 1..17: note = Synthetic construct
  VAR_SEQ 2: note = X is Arg, Thr, Lys, Ala, Asn, Tyr, Gly, Ile, or Leu
  VAR_SEQ 3: note = X is Met, Lys, Arg, Pro, or His
  VAR_SEQ 5..8: note = X is Arg, Thr, Lys, Ala, Asn, Tyr, Gly, Ile, or Leu
  VAR_SEQ 10..11: note = X is Met, Lys, Arg, Pro, or His
  VAR_SEQ 13..14: note = X is Met, Lys, Arg, Pro, or His
  VAR_SEQ 15..16: note = X is Arg, Thr, Lys, Ala, Asn, Tyr, Gly, Ile, or Leu
AXXKXXXXSXXQXXXXS

SEQ ID NO: 15 - protein, 17 aa, synthetic construct
  REGION 1..17: note = Synthetic construct
  VAR_SEQ 2: note = X is Arg, Lys, Asn, Gly, or Ile
  VAR_SEQ 3: note = X is Met, Lys, Arg, Pro. or His
  VAR_SEQ 5..8: note = X is Arg, Lys, Asn, Gly, or Ile
  VAR_SEQ 10..11: note = X is Met, Lys, Arg, Pro. or His
  VAR_SEQ 13..14: note = X is Met, Lys, Arg, Pro. or His
  VAR_SEQ 15..16: note = X is Arg, Lys, Asn, Gly, or Ile
AXXKXXXXSXXQXXXXS

SEQ ID NO: 16 - protein, 17 aa, synthetic construct
  REGION 1..17: note = Synthetic construct
ARMKKNGGSKRQPHIRS

SEQ ID NO: 17 - protein, 12 aa, synthetic construct
  REGION 1..12: note = Synthetic construct.
ATKAYIKSQLGS

SEQ ID NO: 18 - protein, 124 aa, synthetic construct
  REGION 1..124: note = Synthetic construct - Clone 1A7
QVQLLESGGGLVQPGGSLRLSCAASGVKVSTKSMGWVRQAPGKGLEWVSTIARNDGSTYYADSVKGRFTISRDNSKNTLYLQMNSLRAEDTAVYYCARMKKNGGSKRQPHIRSWGQGTLVTVSS

SEQ ID NO: 19 - protein, 119 aa, synthetic construct
  REGION 1..119: note = Synthetic construct - Clone 1F6
QVQLLESGGGLVQPGGSLRLSCAASGFKVTSQAMGWVRQAPGKGLEWVSAIRKGNGSTYYADSVKGRFTISRDNSKNTLYLQMNNLRAEDTAVYYCATKAYIKSQLGSWGQGTLVTVSS

SEQ ID NO: 20 - protein, 25 aa, synthetic construct
  REGION 1..25: note = Synthetic construct
QVQLLESGGGLVQPGGSLRLSCAAS

SEQ ID NO: 21 - protein, 17 aa, synthetic construct
  REGION 1..17: note = Synthetic Construct
MGWVRQAPGKGLEWVST

SEQ ID NO: 22 - protein, 38 aa, synthetic construct
  REGION 1..38: note = Synthetic construct
YYADSVKGRFTISRDNSKNTLYLQMNSLRAEDTAVYYC

SEQ ID NO: 23 - protein, 11 aa, synthetic construct
  REGION 1..11: note = Synthetic construct
WGQGTLVTVSS

SEQ ID NO: 24 - DNA, 372 bp, synthetic construct
  misc_feature 1..372: note = Synthetic construct
  source 1..372: mol_type = other DNA
caggtgcagctgttggagtctgggggaggcttggtacagcctggggggtccctgcgtctctcctgtgcagcctccggagttaaggttagcactaagtctatgggctgggtccgccaggctccagggaagggtctagagtgggtatcaaccattgcgaggaatgacggtagcacatactacgcagactccgtgaagggccggttcaccatctcccgtgacaattccaagaacacgctgtatctgcaaatgaacagcctgcgtgccgaggacaccgcggtatattattgcgcgagaatgaagaagaatggtgggagtaagcgtcagccgcacatcaggtcttggggtcagggaaccctggtcaccgtctcgagc

SEQ ID NO: 25 - DNA, 357 bp, synthetic construct
  misc_feature 1..357: note = Synthetic construct
  source 1..357: mol_type = other DNA
caggtgcagctgttggagtctgggggaggcttggtacagcctggggggtccctgcgtctctcctgtgcagcctccggatttaaggttacctctcaggctatgggctgggtccgccaggctccagggaagggtctagagtgggtatcagccattcggaagggaaacggtagcacatactacgcagactccgtgaagggccggttcaccatctcccgtgacaattccaagaacacgctgtatctgcaaatgaacaacctgcgtgccgaggacaccgcggtatattattgcgcgacaaaggcttatattaagtcgcagttgggctcttggggtcagggaaccctggtcaccgtctcgagc

(19) United States
(12) Patent Application Publication
(10) Pub. No.: US 2025/0019465 A1
(43) Pub. Date: Jan. 16, 2025

(54) COMPOSITIONS AND METHODS FOR RARE HEPARAN SULFATE GLYCAN-TARGETED CANCER TREATMENT

(71) Applicant: THE BOARD OF REGENTS OF THE UNIVERSITY OF TEXAS SYSTEM, Austin, TX (US)

(72) Inventors: Kiyoshi ARIIZUMI, Dallas, TX (US); Jin-Sung CHUNG, Dallas, TX (US); Ponciano D. CRUZ, Jr., Dallas, TX (US)

(73) Assignee: THE BOARD OF REGENTS OF THE UNIVERSITY OF TEXAS SYSTEM, Austin, TX (US)

(21) Appl. No.: 18/709,365

(22) PCT Filed: Nov. 14, 2022
(86) PCT No.: PCT/US22/79827

§ 371 Date: May 10, 2024

Related U.S. Application Data
(60) Provisional application No. 63/279,004, filed on Nov. 12, 2021.

(51) Int. Cl. C07K 16/44; A61K 39/00; A61P 35/00; C07K 16/30

(52) U.S. Cl. CPC C07K 16/44; A61P 35/00; C07K 16/30; A61K 2039/507

(57) ABSTRACT

Provided herein are single domain antibodies (sdAbs) having a specific affinity for a rare heparan sulfate moiety which can be used to disrupt DC-HIL function to reduce and prevent cancer progression and metastasis. Compositions and methods of use thereof are also provided for use of these sdAbs to treat cancer and cancer metastasis in a subject in need thereof.

### CROSS REFERENCE TO RELATED APPLICATION

This application claims the benefit of U.S. Provisional Patent Application No. 63/279,004, filed Nov. 12, 2021, the content of which is incorporated by reference in its entirety.

### ACKNOWLEDGEMENT OF GOVERNMENT SUPPORT

This invention was made with government support under VA Merit Award No. 1|01 BX004069-01 awarded by the United States Department of Veteran Affairs. The government has certain rights in the invention.

### INCORPORATION BY REFERENCE OF THE SEQUENCE LISTING

This application contains a Sequence Listing that has been submitted in XML format via Patent Center and is hereby incorporated by reference in its entirety. The XML copy, created on Nov. 14, 2022, is named “106546-744613_UTSD_3935_Sequence_Listing.xml”, and is 52,000 bytes in size.

### BACKGROUND

### 1. Field

The present inventive concept is directed to single domain antibodies targeting specific heparan sulfate moieties and methods of using the antibodies herein for the treatment of cancer.

### 2. Discussion of Related Art

DC-HIL (also known as gpnmb) receptor binds to diverse receptors and mediate pleiotropic functions including (1) immune suppression via binding to effector T cells (2) tumor angiogenesis via endothelial cells (EC), (3) bone formation and macrophage differentiation via osteoblasts and (4) regulation of cancer stemness via cancer cells. Its natural ligands include heparin sulfate glycans. The present disclosure is based, at least in part, on the surprising discovery that DC-HIL function may be reduced by targeting a rare heparin sulfate glycan and that this may prevent cancer progression and metastasis.

### SUMMARY OF THE INVENTION

The present disclosure is based, at least in part, on the surprising discovery that a rare heparin sulfate glycan plays a role in metastasis of various cancers and that targeting the glycan with a single domain antibody may be sufficient to prevent or reduce cancer progression and metastasis.

Accordingly, various aspects of the instant disclosure are directed to a single domain antibody (sdAb) comprising a variable heavy domain having a specific affinity for a heparan sulfate glycan having a structure of Formula I (GlcNS6S-GlcA-GlcNS6S-IdoA2S-GlcNS6S-IdoA2S-GlcNS6S-GlcA)

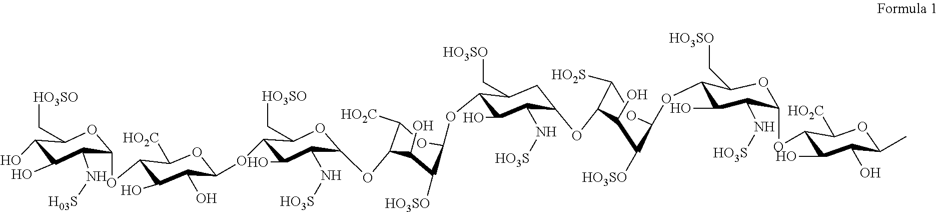

In various embodiments, the single domain antibody of provided herein may comprise a heavy chain complementary determining region 1 (CDRH1) having an amino acid sequence comprising GXKVXXXX, where each X is independently any amino acid (SEQ ID NO: 1). For example, in some embodiments, the sdAb comprises a heavy chain complementary determining region 1 (CDRH1) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2). In further embodiments, the sdAb comprises a heavy chain complementary determining region 1 (CDRH1) having an amino acid sequence having at least 60%, at least 70%, at least 80% or at least 90% sequence identity to GVKVSTKS (SEQ ID NO: 3) or GFKVTSQA (SEQ ID NO: 4). For example, in some embodiments, the sdAb comprises a heavy chain complementary determining region 1 (CDRH1) comprising GVKVSTKS (SEQ ID NO: 3) or GFKVTSQA (SEQ ID NO: 4).

In another aspect, a single domain antibody provided herein may comprise a heavy chain complementary determining region 2 (CDRH2) having an amino acid sequence comprising IXXXXGST, where each X is independently any amino acid (SEQ ID NO: 5). In various embodiments, the sdAb comprises a heavy chain complementary determining region 2 (CDRH2) having an amino acid sequence comprising IXXXXGST, where each X is independently selected from the group consisting of A(Ala), R(Arg), K(Lys), N(Agn), G(Gly), and D (Asp) (SEQ ID NO: 6). For example, the sdAb may comprise a heavy chain complementary determining region 2 (CDRH2) having an amino acid sequence having at least 60%, at least 70%, at least 80% or at least 90% sequence identity to IARNDGST (SEQ ID NO: 7) or IRKGNGST (SEQ ID NO: 8). In some embodiments, the sdAb comprises a heavy chain complementary determining region 2 (CDRH2) having an amino acid sequence comprising IARNDGST (SEQ ID NO: 7) or IRKGNGST (SEQ ID NO: 8).

In another aspect, a single domain antibody provided herein may comprise a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 9) wherein each X1 is independently any amino acid and each X2 is independently absent or is any amino acid. In various aspects, the sdAb may comprise a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 10) wherein each X1 is selected from the group consisting of R (Arg), T(Thr), K(Lys), A(Ala), N(Agn), Y(Tyr), G(Gly), I(Ile), L(Leu) and each X2 is either absent or is selected from the group consisting of M (Met), K(Lys) R(Arg), P(Pro), and H(His). For example, the sdAb may comprise a heavy chain complementary determining region 3 (CDRH3) having an amino acid sequence having at least 60%, at least 70%, at least 80% or at least 90% sequence identity to ARMKKNGGSKRQPHIRS (SEQ ID NO: 16) or ATKAYIKSQLGS (SEQ ID NO: 17). In some embodiments, the sdAb comprises a heavy chain complementary determining region 3 (CDRH3) having an amino acid sequence comprising ARMKKNGGSKRQPHIRS (SEQ ID NO: 16) or ATKAYIKSQLGS (SEQ ID NO: 17).

Accordingly to various aspects of the present disclosure, the single domain antibody (sdAb) provided herein may comprise: (a) a heavy chain complementary determining region 1 (CDRH1) having an amino acid sequence comprising GXKVXXXX, where each X is independently any amino acid (SEQ ID NO: 1); (b) a heavy chain complementary determining region 2 (CDRH2) having an amino acid sequence comprising IXXXXGST, where each X is independently any amino acid (SEQ ID NO: 5); and (c) a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 9) wherein each X1 is independently any amino acid and each X2 is independently absent or is any amino acid.

In another aspect, the sdAb may comprise: (a) a heavy chain complementary determining region 1 (CDRH1) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2); (b) a heavy chain complementary determining region 2 (CDRH2) having an amino acid sequence comprising IXXXXGST, where each X is independently selected from the group consisting of A(Ala), R(Arg), K(Lys), N(Agn), G(Gly), and D (Asp) (SEQ ID NO: 6); and (c) a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 9) wherein each X1 is selected from the group consisting of R (Arg), T(Thr), K(Lys), A(Ala), N(Agn), Y(Tyr), G(Gly), I(lie), L(Leu) and each X2 is either absent or is selected from the group consisting of M (Met), K(Lys) R(Arg), P(Pro), and H(His).

In further aspects, the sdAb may comprise: (a) a heavy chain complementary determining region 1 (CDRH1) having an amino acid sequence comprising GVKVSTKS (SEQ ID NO: 3) or GFKVTSQA (SEQ ID NO: 4); (b) a heavy chain complementary determining region 2 (CDRH2) having an amino acid sequence comprising IARNDGST (SEQ ID NO: 7) or IRKGNGST (SEQ ID NO: 8); and (c) a heavy chain complementary determining region 3 (CDRH3) having an amino acid sequence comprising ARMKKNGGSKRQPHIRS (SEQ ID NO: 16) or ATKAYIKSQLGS (SEQ ID NO: 17).

In another aspect, a single domain antibody is provided comprising: (a) a heavy chain complementary determining region 1 (CDRH1) having an amino acid sequence comprising GVKVSTKS (SEQ ID NO: 3), a heavy chain complementary determining region 2 (CDRH2) having an amino acid sequence comprising IARNDGST (SEQ ID NO: 7) and a heavy chain complementary determining region 3 (CDRH3) having an amino acid sequence comprising ARMKKNGGSKRQPHIRS (SEQ ID NO: 16); or (b) a heavy chain complementary determining region 1 (CDRH1) having an amino acid sequence comprising GFKVTSQA (SEQ ID NO: 4), a heavy chain complementary determining region 2 (CDRH2) having an amino acid sequence comprising IRKGNGST (SEQ ID NO: 8) and a heavy chain complementary determining region 3 (CDRH3) having an amino acid sequence comprising ATKAYIKSQLGS (SEQ ID NO: 17).

In any of the embodiments provided herein, the sdAb can comprise an amino acid sequence having at least 75%, at least 80%, at least 85%, at least 90% or at least 95% sequence identity to SEQ ID NO: 18 or SEQ ID NO: 19. For example, in some embodiments, the sdAb comprises an amino acid sequence comprising SEQ ID NO: 18 or SEQ ID NO: 19.

Another aspect of the instant disclosure is directed to an isolated nucleic acid encoding the single domain antibody provided herein. The isolated nucleic acid may comprise, in some embodiments, at least 85%, at least 90%, or at least 95% sequence identity to SEQ ID NO: 24 or SEQ ID NO: 25. For example, the isolated nucleic acid may comprise SEQ ID NO: 24 or SEQ ID NO: 25.

Further aspects of the instant disclosure are directed to expression vectors containing a nucleic acid encoding the sdAb and host cells thereof.

According to another aspect of the instant disclosure, a pharmaceutical composition is provided comprising a single domain antibody as set forth herein and a pharmaceutically acceptable carrier.

In another aspect, a method of treating a cancer in a subject in need thereof is provided, the method comprising administering the pharmaceutical composition to the subject. In some aspects, the method further comprises preventing, treating, or reducing a metastasis of the cancer in the subject. In some aspects, administering the single domain antibody disrupts formation of premetastatic niches in an organ (e.g., an organ distal to a primary tumor) in the subject. In some embodiments, administering the single domain antibody disrupts rare heparin sulfate (rHS) signaling at a potential premetastatic niche in the subject.

In any of the methods provided herein, the cancer may comprise melanoma, lung cancer, breast cancer, colon cancer, kidney cancer, ovarian cancer, pancreatic cancer, prostate cancer, rectal cancer, stomach cancer, thyroid cancer, uterine cancer, brain tumors or a combination of any thereof.

In any of the methods provided herein, the method may further comprise administering a therapeutically effective amount of an anti-DC-HIL antibody to the subject.

### BRIEF DESCRIPTION OF THE DRAWINGS

Embodiments of the present inventive concept are illustrated by way of example in which like reference numerals indicate similar elements and in which:

FIG. 1A is a table of representative HS oligosaccharides (4-8-mers of HS disaccharide).

FIG. 1B is an illustrative bar graph indicating average binding of DC-HIL to various HS oligosaccharides.

FIG. 1C are representative FACS histograms showing binding of Fc control (blue) or DC-HIL-Fc (red, measured by fluorescently staining with anti-Fc Ab), respectively in SVEC mouse endothelial cells or activated T-cells that were incubated with DC-HIL-Fc (10 mg/ml) plus indicated oligoglycans.

FIG. 2A is a diagram of a phage selection process to identify antibodies according to some aspects of the present disclosure.

FIG. 2B is a schematic of an antibody fragment (e.g., a variable heavy chain, VH, in red) displayed as a fusion with the terminal phage gene Ill protein (green). In accordance with the methods of this disclosure, both proteins were encoded by the phagemid DNA (magnified) and expressed from a common promoter. Also shown is the major gene VIII coat protein (in white).

FIG. 3A-3D show representative dose response curves indicating glycan #12 binding by increasing doses of 1A7 SdAb (FIG. 3A), FGF2 (FIG. 3B) and VEGF (FIG. 3C) or 1A7 SdAb in the presence of DC-HIL-Fc (1 mg/ml) (FIG. 3D, shows % inhibition).

FIG. 3E shows a bar graph showing percent inhibition of either 1A7 SdAb or control SdAb (KLH specific antibody) binding to Glycan #12 by FGF2 or VEGF (at 0.25 or 0.5 mg/ml, respectively).

FIG. 3F shows a representative FACS sorted histogram showing expression of 1A7 epitope on SVEC treated with 50 mM Sodium Cholate (red histogram) or untreated (Blue).

FIG. 3G shows a representative FACS sorted histogram showing DC-HIL-binding on SVEC after pretreatment with 1A7 or control Ab (red and blue histograms, respectively).

FIG. 3H summarizes all data from experiments of binding/inhibition and expression analysis on different tumor lines are summarized in the table. “Block DC-HIL (or VEGF or FGF2)” means blocking of binding to Glycan #12.

FIG. 4 shows a representative histogram showing 1A7 SdAb (10 μg) hybridized with HS glycan microarray consisting of 53 different glycan compounds (Glycan Therapeutics Inc) and fluorescently labeled with anti-c-myc Ab. Hybridized arrays were analyzed for fluorescence intensity (U); average±sd, n=100). SdAb has the C-terminal c-myc tag.

FIG. 5 shows a graph indicating percentages of a T cell population positive for epitopes for 1A7 SdAb, 1F6 SdAb, Syndecan-4 (SD4) or PD-1 as measured by FACS. T cells were isolated from peripheral blood of a healthy donor and incubated in well (2×10⁵ cells/well) with CD2/CD3/CD28-anti-biotin MACS Beads (cell: bead ratio is 1:1.5).

FIG. 6 provides representative images of whole murine lung stained with anti-CD31, 1A7 or 1F6 SdAb (0.5 mg/ml or 10 mg/ml, respectively). ImmPACT Vector Red Substrate was used for color development and followed by counterstaining with hematoxylin. All endothelial cells were stained for CD31, but only some for 1A7 or 1F6.

FIG. 7 provides representative confocal images of a whole ear of untreated mice stained with 1A7 SdAb (shown in red fluorescence) and anti-CD31 Ab (in green, 10 mg/ml and 0.5 mg/ml, respectively). The yellow window is enlarged and shown at the right. 1A7-stains are confined to certain areas in blood vessels.

FIG. 8 shows illustrative images of normal human lung specimens (US Biomax, Inc) stained with anti-CD31 or 1A7 Ab (0.5 mg/ml or 10 mg/ml, respectively). DAB purple substrate was used for color development, followed by hematoxylin counterstaining.

FIGS. 9A and 9B show representative images (FIG. 9A) and plot (FIG. 9B) showing blood vessel growth in mice subcutaneously implanted with matrigel containing DC-HIL-Fc (DCH) or Fc (200 nM) mixed with 10× molecular excess of Glycan #3 or Glycan #12 (See FIG. 1A). Hemoglobulin (Hb) levels were measured in 9B and red-dashed line indicates Hb level in Fc-control plug. Data are shown average±SD, n=4.

FIGS. 10A-10D show representative plots of tumor volume of B16 melanoma cells in mice (FIG. 10A) or RenCa kidney cells in BALB/c mice (FIG. 10C) following intratumoral injection of 1 mg of Glycan #3 (G3) or Glycan #12 (G12, see FIG. 1A) or expression of angiogenic factors by the B16 melanoma cells or RenCa kidney cells measured by qRT-PCR (FIG. 10B and FIG. 10D, respectively).

FIG. 11A shows a representative plot of B16 melanoma tumor cell volume following subcutaneous injection of B16 melanoma cells and i.p. treatment with 1A7 or KLH Ab.

FIG. 11B shows representative FACS data of GFP positive tumor cells isolated from the mice treated in FIG. 11A.

FIGS. 11C-11E provide representative graphs showing number of GFP+ cells in lungs of animals intravenously injected with 1A7 SdAb or KLH SdAb either one day before (d-1) or one day after (d1) intravenous injection with B16 or LL2 cells.

FIGS. 12A-12B show representative FACS plots (FIG. 12A) or summarized data (FIG. 12B) showing number of GFP+ cells in mice injected i.v. with KLH, 1A7 SdAb (10 mg/mouse), anti-DC-HIL (DCH), anti-PDL1 (PL1), anti-VEGFR2 (VER) or combined PL1/VER (PL/VE) mAb (200 mg/mouse) one day before intravenous injection of B16 cells/mouse. GFP+ cells were measured on day 14 following the antibody administration.

FIGS. 13A-13B. SVEC were cultured with 20% of conditioned media (CM) of B16 melanoma cell culture or indicated cytokines (10 ng/ml) or media (None). Next day, cells were harvested and examined by FACS for expression of 1A7 epitope or control (Ctrl) (A) and by qRT-PCR for RNA expression of HS2ST1, HS3ST1, and HS6ST1 genes relative to GAPDH (B). HSST, heparan sulfate sulfo-transferase.

FIG. 14 shows a line graph showing ³H-thymidine incorporation into Pan-T cells isolated from blood sample of a healthy donor incubated with 1A7 or KLH control Ab plus increasing doses of anti-CD3 Ab. After incubation for 2 days, ³H-thymidine was pulsed for 16 h, harvested, and counted for cpm in cells.

FIGS. 15A-15B shows T-cells and monocytic MDSC isolated from blood of patients with colorectal cancer (FIG. 15A) or renal cell carcinoma (FIG. 15B) that were co-cultured (1:1 cell ratio) with anti-CD3/28 Ab plus control (Ctrl) or 3D5 anti-DC-HIL Ab (50 mg/ml) or 1A7 anti-rHS Ab (10 mg/ml) or combination of the last 2 Ab (3D5 and 1A7). IFN-g was assayed 5 days later (median±sd, 3 wells). Results are expressed as % T-cell restoration, with a red-dashed line corresponding to level of IFN-g suppression by MDSC. MDSC from patients A and B showed DC-HIL expression at 60% and 90% of total MDSC, respectively.

FIGS. 16A-16B show representative FACS plot and quantified numbers of GFP+ cells identified in immuno-deficient NSG mice (n=5) that were injected i.v. with KLH or 1A7 SdAb (10 mg/mouse), followed by i.v. injection of 2×10⁵ human SK-MEL-28 melanoma cell line a day later. On Day 14, lung was enumerated by FACS for GFP+ cells (FIG. 16A) and the number in 5×10⁵ lung cells (average±sd, n=5) are summarized (FIG. 16B). *p<0.0001. Data are representative of 2 separate experiments. 1A7 single injection decreased human melanoma in lung by 84%.

FIG. 17A shows a schematic of an animal protocol to treat spontaneous metastasis from a primary subcutaneous tumor using rHS specific sdAbs of the instant disclosure. Briefly, mice were implanted s.c. with LacZ-LL2 cells, 2 weeks later, tumor was resected surgically. Mice were then treated with Ab injection every 3 days. 2 weeks later, lungs were excised and stained with X-gal (labeled LacZ-LL2 cells in blue) and blue stains were counted.

FIG. 17B shows representative lung sections obtained from mice treated as in FIG. 17A and stained with X-gal to label tumor cells (in blue, labeled with green or red arrows) of mice injected subcutaneously with tumor cells

FIG. 17C shows a representative plot quantifying numbers of tumor cells counted per field of view (FOV) in images obtained in FIG. 17B. An average of 5 FOVs is also shown. Each dot represents an individual mouse.

FIG. 18A shows a schematic of a protocol to treat patients using rHS specific sdAbs of the instant disclosure. Briefly, stage III/IV RCC patients (n=39) were treated with immune checkpoint inhibitors (ICIs) and evaluated for tumor response at every follow-up (weeks post-treatment).

FIG. 18B depicts the blood sDC-HIL levels in tumor response of metastatic renal cell carcinoma (mRCC) patients. Stage Ill/IV RCC patients were treated with immune checkpoint inhibitors (ICIs) and evaluated for tumor response at every follow-up (weeks post-treatment) (see FIG. 18A). Tumor response was assessed by Response Evaluation Criteria in Solid Tumours (RECIST); progressive disease (PD), stable disease (SD), partial response (PR) and complete response (CR). At 12 weeks follow-up, patients were categorized into responders (Res, who displayed SD, PR or CR) and non-responders (NR, SD). Initial responders were sorted into two phenotypes; all the time responders (Att Res) who always displayed response to ICIs and acquired resistance (AR) who became unresponsive to ICIs. Non-responders also diverged into two phenotypes; all the time non-responders (Att NR) who never exhibited response and acquired sensitivity (AS) who acquired response to ICIs. Changes over time in sDC-HIL (ng/ml) are plotted with tumor response of representative patients of each cohort.

FIG. 19 shows the follow-up studies with the 39 patients from FIGS. 18A-18B. Red dotted lines show the average sDC-HIL levels among initial responders.

FIGS. 20A-20B show that rHS Ab treatment inhibited tumor growth of B16 mouse melanoma more than PD-1 Ab. In FIG. 20A B16F10 melanoma cells were implanted s.c. into B57BL/6 mice (n=5). Six days later, mice were treated with i.p. injection of Ab every 2 days; 1A7 rHS Ab (20 mg/mouse), PD1 (200 mg/mouse), rHS/PD1 (20 mg and 200 mg, respectively) or control (Ctrl) Ab (200 mg/mouse). Tumor volume was measured every 2 days (FIG. 20A). On day 15, all mice were euthanized, tumors were excised and IHC-stained with marker Ab (CD4, CD8, CD11b, and CD31) (FIG. 20B). Positive staining is assayed by % positively stained areas in 4.5 mm² view (mean±sd, n=5). * significant to control using 2 way-ANOVA. Ns means not significant.

FIGS. 21A-21B depict stimulation of rHS-bearing receptors on human T cells by rHS Ab attenuates CD3-proliferation response significantly more than PD1 receptor by specific Ab. CD3 T cells isolated from peripheral blood of healthy donors (HD) were cultured with anti-CD3 Ab and increasing doses (mM) of test Ab (control (Ctrl) IgG, PD1 Ab (Keytruda, pembrolizumab), 1A7 and 1F6 rHS single domain Ab), and proliferation was measured by 3H-thymidine incorporation (cpm) into T cells (FIG. 21A). Each Ab was assessed for the dose (mM) required for 50% inhibition of 3H-cpm in control culture with no test Ab; IC50 value (FIG. 21B).

FIG. 22A depicts the experimental design to evaluate efficacy of test Ab to inhibit tumor growth of human melanoma SK-MEL-28.

FIG. 22B shows a line graph showing tumor volume every two days for mice injected with human melanoma cells (see FIG. 22A).

FIG. 22C shows bar graphs showing % leukocyte subpopulation among CD45+ human cells. Two-way ANOVA statistical analysis was performed between groups. P values heighted in red and black fonts are significant and insignificant, respectively.

FIGS. 23A-23C show treatment of human melanoma SK-MEL-5 by anti-DC-HIL/rHS Ab vs. PD1 Keytruda in PBMC-humanized mice. FIG. 23A is a line graph showing tumor growth curves of SK-MEL-5 on mice treated with control Ab, 3D5, PD1 Keytruda, 3D5/rHS, or a mixture of all Ab. FIG. 23B shows images of the tumors excised on day 16. FIG. 23C is a bar graph showing the tumor weight in grams.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the surprising discovery of a new single domain antibodies (SDAb) that disrupt formation of premetastatic niches in distal organs by targeting rare components of heparan sulfate glycans, which are the ligands of DC-HIL/glycoprotein nmb (Gpnmb), a receptor expressed on antigen presenting cells and MDSC. The present disclosure provides for antibodies and pharmaceutical compositions thereof, and methods of treating cancer using the antibodies and pharmaceutical compositions disclosed herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mM includes all values from 1 mM to 9 mM, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “modify” or “modifying” and grammatical variations thereof, when used in reference to any of the compositions (e.g., proteins, protein domains, peptides, peptide fragments, polypeptide sequences) disclosed herein means that the modified composition deviates from a reference composition.

The term “recombinant” as used herein refers to an introduction of a heterologous nucleic acid or amino acid, and/or the alteration of a native nucleic acid or protein.

The term “solid support” as used herein refers to a material having a rigid or semi-rigid surface. Such materials will preferably take the form of small beads, pellets, disks, chips, or wafers, although other forms may be used.

The term “surface” as used herein refers to any generally two-dimensional structure on a solid substrate and may have steps, ridges, kinks, terraces, and the like without ceasing to be a surface.

### II. Antibodies

The present disclosure provides for an antibody (a “rHS antibody” or “anti-rHS antibody) that specifically binds to a rare heparin sulfate glycan. In some aspects, the rare heparin sulfate glycan comprises a structure of Formula I (GlcNS6S-GlcA-GlcNS6S-IdoA2S-GlcNS6S-IdoA2S-GlcNS6S-GlcA):

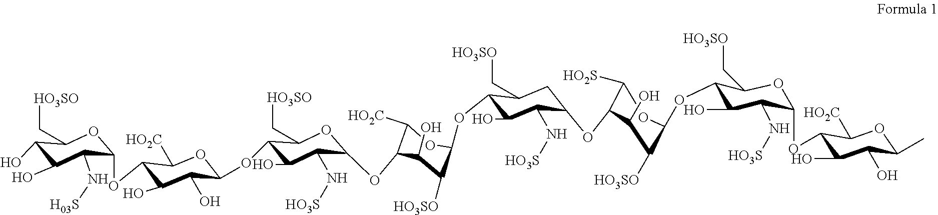

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as single domain antibodies, Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

Any of the rHS antibodies provided herein may be single domain antibodies. In some embodiments, the rHS antibodies are single domain antibodies comprising a single variable heavy region.

A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, AI-lazikani et al (1997) J. Molec. Biol. 273:927-948; and Almagro, J. Mol. Recognit. 17:132-143 (2004). See also hgmp.mrc.ac.uk and bioinf.org.uk/abs.

In some embodiments, an rHS antibody or antigen binding fragment as described herein has a suitable binding affinity for the target antigen (e.g., a rare heparin sulfate glycan having the structure of Formula I) or antigenic epitopes thereof. As used herein, “binding affinity” refers to the apparent association constant or KA. The KA is the reciprocal of the dissociation constant (KD). The rHS antibody described herein may have a binding affinity (KD) of at least 10-5, 10-6, 10-7, 10-8, 10-9, 10-10 M, or lower for the target antigen or antigenic epitope. An increased binding affinity corresponds to a decreased KD. Higher affinity binding of an antibody for a first antigen relative to a second antigen can be indicated by a higher KA (or a smaller numerical value KD) for binding the first antigen than the KA (or numerical value KD) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 1,000,000 fold. In some embodiments, any of the rHS antibodies or antigen binding fragment may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the rHS antibody provided herein may be derived from single domain antibody clones 1A7 or 1F6, each comprising a single variable heavy chain (V_{H}), provided below as SEQ ID NOs: 18 and 19 with CDRs in boldface (determined by IMGT/DomainGapAlign program).

Clone 1A7-
SEQ ID NO: 18
QVQLLESGGGLVQPGGSLRLSCAASGVKVSTKSMGWVRQAPGKGLEWVS
TIARNDGSTYYADSVKGRFTISRDNSKNTLYLQMNSLRAEDTAVYYCAR
MKKNGGSKRQPHIRSWGQGTLVTVSS
Clone 1F6-
SEQ ID NO: 19
QVQLLESGGGLVQPGGSLRLSCAASGFKVTSQAMGWVRQAPGKGLEWVS
AIRKGNGSTYYADSVKGRFTISRDNSKNTLYLQMNNLRAEDTAVYYCAT
KAYIKSQLGSWGQGTLVTVSS

The rHS antibodies derived from reference antibodies 1A7 or 1F6 may comprise substantially similar heavy chain complementary regions (CDRs), which can be grafted into a suitable V_{H} framework. An antibody having “substantially similar” heavy chain CDRs relative to the corresponding CDRs in a reference antibody means that the heavy chain CDRs in the antibody, in collection, contain less than 10 amino acid residue variations (e.g., less than 9, 8, 7, 6, 5, 4, 3, 2, or 1 amino acid variation) relative to the corresponding CDRs, in collection, in the reference antibody. For example, the rHS antibody may comprise only up to 8 (e.g., 8, 7, 6, 5, 4, 3, 2, or 1) amino acid residue variations in the total heavy chain CDR regions and binds the same epitope of rHS with substantially similar affinity (e.g., having a K_{D} value in the same order) as the reference antibody.

In some embodiments, the rHS antibodies provided herein may comprise a heavy chain complementary determining region 1 (CDRH1) defined by a consensus sequence of GXKVXXXX, where each X is any amino acid (SEQ ID NO: 1). In some embodiments, the rHS antibodies provided herein may comprise a CDRH1 comprising a consensus sequence of GXKVXXXX, where each X is independently selected from the group consisting of V(Val), F(Phe), S(Ser), T(Tyr), K(Lys), Q(Gln), and A(Ala) (SEQ ID NO: 2).

In some embodiments, the rHS antibodies provided herein may comprise a CDRH1 having at least 60%, at least 70%, at least 80% or at least 90% sequence identity to GVKVSTKS (SEQ ID NO: 3) or GFKVTSQA (SEQ ID NO: 4). In some embodiments, the rHS antibodies provided herein may comprise a CDRH1 comprising SEQ ID NO: 3 or SEQ ID NO: 4. In some embodiments, the rHS antibodies comprise a CDRH1 that is substantially similar to SEQ ID NO: 3 or SEQ ID NO: 4.

In various aspects, the rHS antibodies may comprise a heavy chain complementary determining region 2 (CDRH2) comprising a consensus sequence comprising IXXXXGST, wherein each X is any amino acid (SEQ ID NO: 5). In some embodiments, the rHS antibodies comprise a CDRH2 having an amino acid sequence comprising IXXXXGST, wherein each X is is independently selected from the group consisting of A(Ala), R(Arg), K(Lys), N(Agn), G(Gly), and D (Asp) (SEQ ID NO: 6).

In some embodiments, the rHS antibodies may comprise a CDRH2 having at least 60%, at least 70%, at least 80% or at least 90% sequence identity to IARNDGST (SEQ ID NO: 7) or IRKGNGST (SEQ ID NO: 8). In some embodiments, the rHS antibodies provided herein may comprise a CDRH2 having an amino acid sequence comprising IARNDGST (SEQ ID NO: 7) or IRKGNGST (SEQ ID NO: 8).

In various aspects, the rHS antibodies may comprise a heavy chain complementary determining region 3 (CDRH3) comprising a consensus sequence comprising A-X1-X2-K-X1-X1-X1-X1-S-X2-X2-Q-X2-X2-X1-X1-S(SEQ ID NO: 9), wherein each X1 is independently any amino acid and each X2 is either absent or any amino acid. In some embodiments, the rHS antibody may comprise a CDRH3 comprising a consensus sequence comprising A-X1-X2-K-X1-X1-X1-X1-S-X2-X2-Q-X2-X2-X1-X1-S each X1 is selected from the group consisting of R (Arg), T(Thr), K(Lys), A(Ala), N(Agn), Y(Tyr), G(Gly), I(lie), L(Leu) and each X2 is either absent or is selected from the group consisting of M (Met), K(Lys) R(Arg), P(Pro), and H(His) (SEQ ID NO: 10).

In various embodiments, the rHS antibody may comprise a CDRH3 comprising a consensus sequence comprising A-X1-K-X1-X1-X1-X1-S-Q-X1-X1-S, wherein each X1 is an amino acid (SEQ ID NO: 11). In various embodiments, the rHS antibody may comprise a CDRH3 comprising a consensus sequence comprising A-X1-K-X1-X1-X1-X1-S-Q-X1-X1-S, wherein each X1 is R, T, K, A, N, Y, G, I, or L (SEQ ID NO: 12). In some embodiments, the rHS antibody may comprise a CDRH3 comprising a consensus sequence comprising A-X1-K-X1-X1-X1-X1-S-Q-X1-X1-S, wherein each X1 is T(Thr), A(Ala), Y(Tyr), I(lie), K(Lys), L(Leu), and G(Gly) (SEQ ID NO: 13).

In various embodiments, the rHS antibody may comprise a CDRH3 comprising a consensus sequence comprising A-X1-X2-K-X1-X1-X1-X1-S-X2-X2-Q-X2-X2-X1-X1-S, wherein each X1 is R, T, K, A, N, Y, G, I, or L and each X2 is selected from the group consisting of M (Met), K(Lys) R(Arg), P(Pro), and H(His) (SEQ ID NO: 14). In various embodiments, the rHS antibody may comprise a CDRH3 comprising a consensus sequence comprising A-X1-X2-K-X1-X1-X1-X1-S-X2-X2-Q-X2-X2-X1-X1-S, wherein each X1 is selected from the group consisting of R (Arg), T(Thr), K(Lys), A(Ala), N(Asn), Y(Tyr), G(Gly), I(Ile), L(Leu) and each X2 is selected from the group consisting of M (Met), K(Lys) R(Arg), P(Pro), and H(His) (SEQ ID NO: 15). In any of these embodiments, X1 may be selected from the group consisting of T(Thr), A(Ala), Y(Tyr), I(Ile), K(Lys), L(Leu), and G(Gly). In any of these embodiments, X1 may be selected from the group consisting of R(Arg), K(Lys), N(Gln), G(Gly), and I(Ile).

In various embodiments, the rHS antibodies may comprise a CDRH3 having at least 60%, at least 70%, at least 80% or at least 90% sequence identity to ARMKKNGGSKRQPHIRS (SEQ ID NO: 16) or ATKAYIKSQLGS (SEQ ID NO: 17). In some embodiments, the rHS antibodies comprise a CDRH3 having an amino acid sequence comprising ARMKKNGGSKRQPHIRS (SEQ ID NO: 16) or ATKAYIKSQLGS (SEQ ID NO: 17). In some embodiments, the rHS antibodies comprise a CDRH3 having an amino acid sequence that consists or consists essentially of ARMKKNGGSKRQPHIRS (SEQ ID NO: 16) or ATKAYIKSQLGS (SEQ ID NO: 17).

For ease of reference, consensus sequences and exemplary CDR sequences that may be used in an rHS antibody of the present disclosure are provided in the table below.

| CDR | Sequence | SEQ ID NO: |
|---|---|---|
| CDR_{H1} | GXKVXXXX, X = any amino acid | 1 |
| CDR_{H1} | GXKVXXXX, X is V(Val), F (Phe), S (Ser), T (Thr), K | 2 |
| | (Lys), Q (Gln), or A (Ala) | |
| CDR_{H1} | GVKVSTKS | 3 |
| CDR_{H1} | GFKVTSQA | 4 |
| CDR_{H2} | IXXXXGST; X = any amino acid | 5 |
| CDR_{H2} | IXXXXGST; X = A, R, K, N, G, D | 6 |
| CDR_{H2} | IARNDGST | 7 |
| CDR_{H2} | IRKGNGST | 8 |
| CDR_{H3} | A-X1-X2-K-X1-X1-X1-X1-S-X2-X2-Q-X2-X2-X1-X1-S, | 9 |
| | Each X1 is independently any amino acid; each X2 is | |
| | independently absent or any amino acid | |
| CDR_{H3} | A-X1-X2-K-X1-X1-X1-X1-S-X2-X2-Q-X2-X2-X1-X1-S; | 10 |
| | X1 is R, T, K, A, N, Y, G, I, L; X2 is absent or M, K, | |
| | R, P, H | |
| CDR_{H3} | A-X1-K-X1-X1-X1-X1-S-Q-X1-X1-S, each X1 is any | 11 |
| | amino acid. | |
| CDR_{H3} | A-X1-K-X1-X1-X1-X1-S-Q-X1-X1-S, each X1 is R, T, | 12 |
| | K, A, N, Y, G, I, L | |
| CDR_{H3} | A-X1-K-X1-X1-X1-X1-S-Q-X1-X1-S, each X1 is T(Thr), | 13 |
| | A(Ala), Y(Tyr), I(Ile), K(Lys), L(Leu), and G(Gly). | |
| CDR_{H3} | A-X1-X2-K-X1-X1-X1-X1-S-X2-X2-Q-X2-X2-X1-X1-S; | 14 |
| | wherein each X1 is R, T, K, A, N, Y, G, I, L; and X2 | |
| | is M, K, R, P, or H | |
| CDR_{H3} | A-X1-X2-K-X1-X1-X1-X1-S-X2-X2-Q-X2-X2-X1-X1-S; | 15 |
| | wherein each X1 is R(Arg), K(Lys), N(Asn), G(Gly), and | |
| | I(Ile); and X2 is M, K, R, P, or H | |
| CDR_{H3} | ARMKKNGGSKRQPHIRS | 16 |
| CDR_{H3} | ATKAYIKSQLGS | 17 |

In any of the embodiments described herein, the rHS antibody may comprise human variable heavy chain framework region. For example, the human framework region may comprise V3-23/D47. In some aspects, the human framework region may comprise an amino acid sequence having at least 60%, at least 70%, at least 80%, or at least 90% sequence identity to any one of SEQ ID NOs: 20 to 23, provided in the table below as FR1, FR2, FR3, and FR4.

| Framework | | SEQ ID |
|---|---|---|
| region | Sequence | NO: |
| FR1 | QVQLLESGGGLVQPGGSLRLSCAAS | 20 |
| FR2 | MGWVRQAPGKGLEWVST | 21 |
| FR3 | YYADSVKGRFTISRDNSKNTLYLQMNSLRAEDTAVYYC | 22 |
| FR4 | WGQGTLVTVSS | 23 |

The exemplary CDR sequences and framework region sequences described above may be combined in various ways to generate an rHS antibody having high affinity and specificity towards a heparin sulfate having a structure of Formula I. Various exemplary combinations of CDR sequences are provided below. The following list is not exhaustive and not limiting and further combinations may be envisioned by one of skill in the art.

In various embodiments, the rHS antibody may comprise a) a heavy chain complementary determining region 1 (CDRH1) having an amino acid sequence comprising GXKVXXXX, where each X is independently any amino acid (SEQ ID NO: 1); (b) a heavy chain complementary determining region 2 (CDRH2) having an amino acid sequence comprising IXXXXGST, where each X is independently any amino acid (SEQ ID NO: 5); and
(c) a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 9) wherein each X1 is independently any amino acid and each X2 is independently absent or is any amino acid.

In various embodiments, the rHS antibody may comprise a (a) a heavy chain complementary determining region 1 (CDRH1) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2); (b) a heavy chain complementary determining region 2 (CDRH2) having an amino acid sequence comprising IXXXXGST, where each X is independently selected from the group consisting of A(Ala), R(Arg), K(Lys), N(Asn), G(Gly), and D (Asp) (SEQ ID NO: 6); and (c) a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 9) wherein each X1 is selected from the group consisting of R (Arg), T(Thr), K(Lys), A(Ala), N(Asn), Y(Tyr), G(Gly), I(Ile), L(Leu) and each X2 is either absent or is selected from the group consisting of M (Met), K(Lys) R(Arg), P(Pro), and H(His).

In still further embodiments, the rHS antibody may comprise (a) a heavy chain complementary determining region 1 (CDRH1) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2); (b) a heavy chain complementary determining region 2 (CDRH2) having an amino acid sequence comprising IXXXXGST, where each X is independently selected from the group consisting of A(Ala), R(Arg), K(Lys), N(Asn), G(Gly), and D (Asp) (SEQ ID NO: 6); and (c) a heavy chain complementary determining region 3 (CDRH3) having an amino acid sequence comprising A-X1-K-X1-X1-X1-X1-S-Q-X1-X1-S(SEQ ID NO: 12) wherein each X1 is selected from the group consisting of R (Arg), T(Thr), K(Lys), A(Ala), N(Asn), Y(Tyr), G(Gly), I(Ile), L(Leu). In some aspects, X1 may be selected from the group consisting of (Thr), A(Ala), Y(Tyr), I(Ile), K(Lys), L(Leu), and G(Gly) (SEQ ID NO: 13).

In various embodiments, the rHS antibody may comprise (a) a heavy chain complementary determining region 1 (CDRH1) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2); (b) a heavy chain complementary determining region 2 (CDRH2) having an amino acid sequence comprising IXXXXGST, where each X is independently selected from the group consisting of A(Ala), R(Arg), K(Lys), N(Asn), G(Gly), and D (Asp) (SEQ ID NO: 6); and (c) a heavy chain complementary determining region 3 (CDRH3) having an amino acid sequence comprising A-X1-X2-K-X1-X1-X1-X1-S-X2-X2-Q-X2-X2-X1-X1-S(SEQ ID NO: 14) wherein each X1 is independently selected from the group consisting of R (Arg), T(Thr), K(Lys), A(Ala), N(Asn), Y(Tyr), G(Gly), I(Ile), L(Leu) and each X2 is independently selected from the group consisting of M (Met), K(Lys) R(Arg), P(Pro), and H(His). In some embodiments, each X1 is independently selected from the group consisting of R(Arg), K(Lys), N(Asn), G(Gly), and I(Ile) (SEQ ID NO: 15).

In still further embodiments, the rHS antibody may comprise (a) a heavy chain complementary determining region 1 (CDRH1) having an amino acid sequence having at least 60%, at least 70%, at least 80% or at least 90% sequence identity to GVKVSTKS (SEQ ID NO: 3) or GFKVTSQA (SEQ ID NO: 4); (b) a heavy chain complementary determining region 2 (CDRH2) having an amino acid sequence having at least 60%, at least 70%, at least 80% or at least 90% sequence identity to IARNDGST (SEQ ID NO: 7) or IRKGNGST (SEQ ID NO: 8); and (c) a heavy chain complementary determining region 3 (CDRH3) having an amino acid sequence having at least 60%, at least 70%, at least 80% or at least 90% sequence identity to ARMKKNGGSKRQPHIRS (SEQ ID NO: 16) or ATKAYIKSQLGS (SEQ ID NO: 17).

In still further embodiments, the rHS antibody may comprise (a) a heavy chain complementary determining region 1 (CDRH1) having an amino acid sequence comprising GVKVSTKS (SEQ ID NO: 3) or GFKVTSQA (SEQ ID NO: 4); (b) a heavy chain complementary determining region 2 (CDRH2) having an amino acid sequence comprising IARNDGST (SEQ ID NO: 7) or IRKGNGST (SEQ ID NO: 8); and (c) a heavy chain complementary determining region 3 (CDRH3) having an amino acid sequence comprising ARMKKNGGSKRQPHIRS (SEQ ID NO: 16) or ATKAYIKSQLGS (SEQ ID NO: 17).

In various embodiments, the rHS antibody may comprise (a) a heavy chain complementary determining region 1 (CDRH1) having an amino acid sequence comprising GVKVSTKS (SEQ ID NO: 3), a heavy chain complementary determining region 2 (CDRH2) having an amino acid sequence comprising IARNDGST (SEQ ID NO: 7) and a heavy chain complementary determining region 3 (CDRH3) having an amino acid sequence comprising ARMKKNGGSKRQPHIRS (SEQ ID NO: 16); or (b) a heavy chain complementary determining region 1 (CDRH1) having an amino acid sequence comprising GFKVTSQA (SEQ ID NO: 4), a heavy chain complementary determining region 2 (CDRH2) having an amino acid sequence comprising IRKGNGST (SEQ ID NO: 8) and a heavy chain complementary determining region 3 (CDRH3) having an amino acid sequence comprising ATKAYIKSQLGS (SEQ ID NO: 17).

In various embodiments, the rHS antibody may comprise an amino acid sequence having at least 75%, at least 80%, at least 85%, at least 90% or at least 95% sequence identity to SEQ ID NO: 18 or SEQ ID NO: 19. For example, in some embodiments, the rHS antibody may comprise, consist, or consist essentially of SEQ ID NO: 18 or SEQ ID NO: 19.

In any of the embodiments provided herein, conservative amino acid substitutions may be made. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### III. Method of Making Antibodies

In some examples, a rHS antibody as disclosed herein can be prepared by recombinant technology as exemplified below. It is noted that while single domain antibodies (e.g., comprising a single variable heavy chain), full size antibodies are also envisioned that may be prepared by combining exemplary variable heavy chains provided herein with suitable light chains and/or constant regions.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

Accordingly, in some embodiments, suitable nucleic acid sequences that may encode one or more of the chains of the rHS antibodies described herein are provided. In various aspects, the nucleic acids encode the single chain rHS antibodies provided herein (e.g., comprising a single heavy variable region). In some embodiments, the nucleic acid comprise a nucleic acid sequence having at least 85%, at least 90%, or at least 95% sequence identity to SEQ ID NO: 24 or SEQ ID NO: 25. In some embodiments, the nucleic acids comprise SEQ ID NO: 24 or SEQ ID NO: 25.

SEQ ID NO: 24
CAGGTGCAGCTGTTGGAGTCTGGGGGAGGCTTGGTACAGCCTGGGGGGT
CCCTGCGTCTCTCCTGTGCAGCCTCCGGAGTTAAGGTTAGCACTAAGTC
TATGGGCTGGGTCCGCCAGGCTCCAGGGAAGGGTCTAGAGTGGGTATCA
ACCATTGCGAGGAATGACGGTAGCACATACTACGCAGACTCCGTGAAGG
GCCGGTTCACCATCTCCCGTGACAATTCCAAGAACACGCTGTATCTGCA
AATGAACAGCCTGCGTGCCGAGGACACCGCGGTATATTATTGCGCGAGA
ATGAAGAAGAATGGTGGGAGTAAGCGTCAGCCGCACATCAGGTCTTGGG
GTCAGGGAACCCTGGTCACCGTCTCGAGC
SEQ ID NO: 25
CAGGTGCAGCTGTTGGAGTCTGGGGGAGGCTTGGTACAGCCTGGGGGGT
CCCTGCGTCTCTCCTGTGCAGCCTCCGGATTTAAGGTTACCTCTCAGGC
TATGGGCTGGGTCCGCCAGGCTCCAGGGAAGGGTCTAGAGTGGGTATCA
GCCATTCGGAAGGGAAACGGTAGCACATACTACGCAGACTCCGTGAAGG
GCCGGTTCACCATCTCCCGTGACAATTCCAAGAACACGCTGTATCTGCA
AATGAACAACCTGCGTGCCGAGGACACCGCGGTATATTATTGCGCGACA
AAGGCTTATATTAAGTCGCAGTTGGGCTCTTGGGGTCAGGGAACCCTGG
TCACCGTCTCGAGC

In some embodiments, a nucleic acid encoding a single domain antibody provided herein is cloned into a suitable expression vector operably linked to a promoter. In some embodiments, one or more nucleic acids are cloned into one or more expression vectors, each nucleic acid encoding, for example, a heavy chain variable domain described herein and, optionally, a light chain, a light chain variable domain, a constant region (Fc) or any combination thereof.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes at least the heavy chain of an rHS antibody (including a single domain antibody), as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the one or more polypeptide chains that form the antibody (e.g., a single domain antibody which comprises a single polypeptide chain, or a full antibody which comprises two polypeptide chains), which can be recovered from the cells or from the culture medium. When necessary, two chains recovered from the host cells can be incubated under suitable conditions allowing for the formation of the antibody.

In one example, one recombinant expression vector is provided, encoding the variable heavy chain domain rHS antibody provided herein. The recombinant expression vector can be introduced into a suitable host cell (e.g., dhfr-CHO cell) by a conventional method, e.g., calcium phosphate-mediated transfection. Alternatively, the expression vector can be introduced into suitable host cells. Positive transformants can be selected and cultured under suitable conditions allowing for the expression of the polypeptide chain of the antibody. Once expressed, the rHS single domain antibody may be recovered from the host cells or from the culture medium.

In various embodiments, various antibodies that comprise the variable heavy chain domain described herein may be made. These antibodies may include some or all of a heavy chain and/or a light chain. In this case, when more than one polypeptide is required, more than one expression vectors may be provided. For example, one expression vector may comprise a nucleic acid sequence encoding a heavy chain and the other expression vector comprises a nucleic acid sequence encoding a light chain. These two vectors may be introduced into the same or different host cells according to the methods above. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, a light chain, or both of an rHS antibody as described herein, vectors (e.g., expression vectors) containing such; and host cells comprising the vectors are within the scope of the present disclosure. For example, nucleic acids encoding the heavy chain (e.g., the variable heavy chain domain) of an rHS antibody described herein, vectors (e.g., expression vectors) containing such; and host cells comprising the vectors are within the scope of the present disclosure.

rHS antibodies thus prepared can be characterized using methods known in the art, whereby a suppression in T cell activation and angiogenesis is detected and/or measured. For example, an ELISA-type assay may be suitable for qualitative or quantitative measurement of DC-HIL, Ab titers to rHS. Other methods include in vitro T cell activation assays; co-culture assays of T cells and MDSC, antigen presenting assays, in vivo matrigel plug assays for angiogenesis, tumor growth assays, experimental lung metastasis assays, spontaneous lung metastasis assay. These and other assays are known in the art and can be found in, for example, Pouliot N et al., “Investigating Metastasis Using In Vitro Platforms.” (In: Madame Curie Bioscience Database [Internet]. Austin (TX): Landes Bioscience; 2000-2013) which is incorporated herein by reference in its entirety.

### IV. Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 m, particularly 0.1 and 0.5 m, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### V. Therapeutic Methods

Various aspects of the present disclosure provide methods of treating a disease, for example a cancer, by administering a therapeutically effective amount of an rHS antibody (e.g., a single domain rHS antibody). The antibody may be administered in a pharmaceutical composition as described above.

In various aspects, the methods provided herein further comprise preventing, treating or reducing a metastasis of the cancer in the subject. For example, in some embodiments, the single domain antibody disrupts formation of premetastatic niches in an organ in the subject. In some cases, the organ is distal to a primary tumor in the subject (e.g., is a site or potential site of metastasis of the primary cancer). In some embodiments, administering the antibodies described herein disrupts rare heparin sulfate (rHS) signaling at a potential premetastatic niche in the subject.

In any of the embodiments, herein the cancer may comprise melanoma, lung cancer, breast cancer, colon cancer, kidney cancer, ovarian cancer, pancreatic cancer, prostate cancer, rectal cancer, stomach cancer, thyroid cancer, uterine cancer, brain tumors or any combination thereof. In various embodiments, the cancer may be a cancer prone to, or at high risk of, metastasis.

In some aspects, the methods of treating a disease provided herein comprise administering a therapeutically effective amount of an rHS antibody (e.g., a single domain rHS antibody) and a therapeutically effective amount of an anti-DC HIL antibody. The antibodies may be administered together or separately. The antibodies may be administered at the same time, sequentially, concurrently, or separately with a period of time between the separate administrations. The antibodies may be administered in a pharmaceutical composition as described above. The antibodies may be administered together in the same pharmaceutical composition or in separate pharmaceutical compositions. The anti-DC HIL antibody may be an anti-DC HIL antibody as described in U.S. Pat. No. 10,517,948, which is hereby incorporated by reference in its entirety.

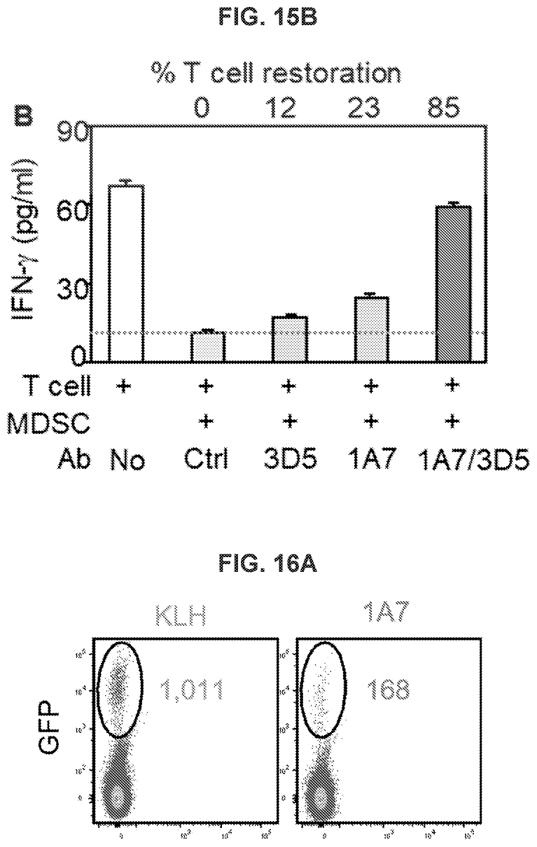

In some aspects, the administration of a therapeutically effective amount of an anti-rHS antibody (e.g., a single domain rHS antibody and a therapeutically effective amount of an anti-DC HIL antibody results in a therapeutic effect that is greater than the administration of either antibody (e.g., rHS antibody or anti-DC HIL antibody) alone. By way of a non-limiting example, administration of both antibodies may restore IFN-γ expression to levels greater than those by each individual antibody and exhibit an increased percentage of T cell restoration. (See, e.g., FIG. 15B).

As used herein, “an effective amount” refers to the amount of each active agent required to confer therapeutic effect on the subject, either alone or in combination with one or more other active agents. Determination of whether an amount of the antibodies disclosed herein achieved a therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In some embodiments, a maximum dose of the individual components or combinations thereof may be used, that is, the highest safe dose according to sound medical judgment.

In some embodiments, empirical considerations, such as the half-life, generally will contribute to the determination of the dosage. In some embodiments, antibodies herein that are compatible with the human immune system, such as humanized antibodies, may be used to prolong half-life of the antibody and/or to prevent the antibody from being attacked by the host's immune system. In some embodiments, frequency of administration may be determined and adjusted over the course of therapy, and is generally, but not necessarily, based on treatment and/or suppression and/or amelioration and/or delay of a target disease/disorder. In some embodiments, sustained continuous release formulations of an antibody herein may be appropriate. Various formulations and devices for achieving sustained release are known in the art.

In some embodiments, dosages for an antibody as described herein may be determined empirically in individuals who have been given one or more administration(s) an antibody disclosed herein. In accordance with some embodiments herein, individuals can be given incremental dosages of the antibody. To assess efficacy of the antibody, an indicator of the disease/disorder can be followed.

In some embodiments, for administration of any of the antibodies described herein, an initial candidate dosage can be about 2 mg/kg. For the purpose of the present disclosure, a typical daily dosage may range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, treatment methods of the present disclosure may be sustained until a desired suppression of symptoms occurs and/or until sufficient therapeutic levels are achieved to alleviate a target disease or disorder, or a symptom thereof. In some embodiments, a dosing regimen herein may comprise administering an initial dose of about 2 mg/kg antibody, followed by a weekly maintenance dose of about 1 mg/kg of antibody, or followed by a maintenance dose of about 1 mg/kg antibody every other week. In some embodiments, other dosage regimens may be useful, depending on the pattern of pharmacokinetic decay that the practitioner wishes to achieve. In accordance with some embodiments herein, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) antibody may be used. In some embodiments, dosing frequency may be once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, the progress of this therapy can be easily monitored by conventional techniques and assays. In some embodiments, a dosing regimen (including the disclosed antibody used) suitable for use herein can vary over time.

In some embodiments, for an adult patient of normal weight, doses ranging from about 0.3 to about 5.00 mg/kg antibody may be administered. In some embodiments, the dosage of the antibody described herein can be about 10 mg/kg. The particular dosage regimen, i.e., dose, timing and repetition, can depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific peptides (or compositions thereof) employed, the type and severity of the disease/disorder, whether the antibody is administered for preventive or therapeutic purposes, previous therapy, the patient's clinical history and response to the antibody, an/or the discretion of the attending physician. In some embodiments, a clinician will administer one or more of the disclosed antibodies until a dosage is reached that achieves the desired result. In some embodiments, for example, the desired result may be a decrease in the severity or a complete treatment of a cancer. Methods of determining whether a dosage resulted in the desired result would be evident to one of skill in the art. In some embodiments, administration of one or more antibodies herein can be continuous or intermittent, depending, for example, upon the recipient's physiological condition, whether the purpose of the administration is therapeutic or prophylactic, and other factors known to skilled practitioners. In some embodiments, administration of an antibody herein may be essentially continuous over a preselected period of time or may be in a series of spaced dose, e.g., either before, during, or after developing a target disease or disorder.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1—Glycan #12 (rHS) is the Relevant Ligand of DC-HIL

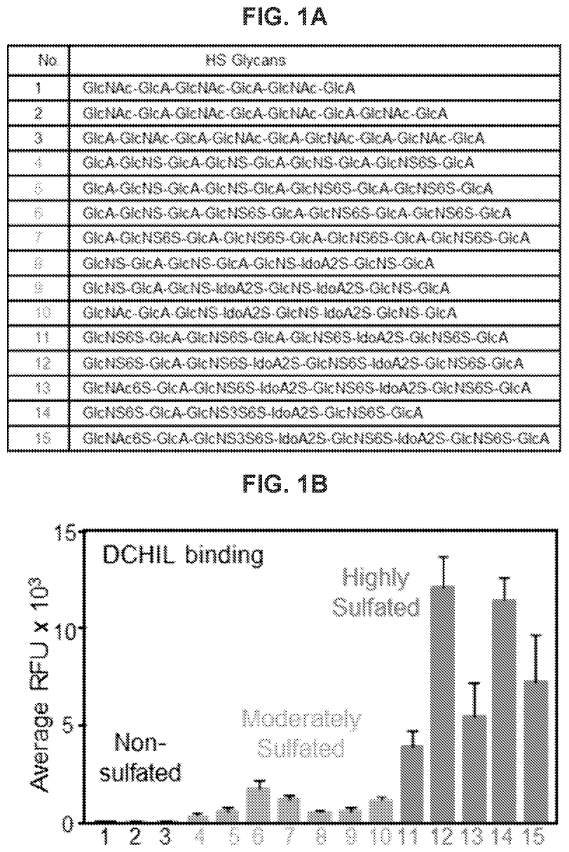

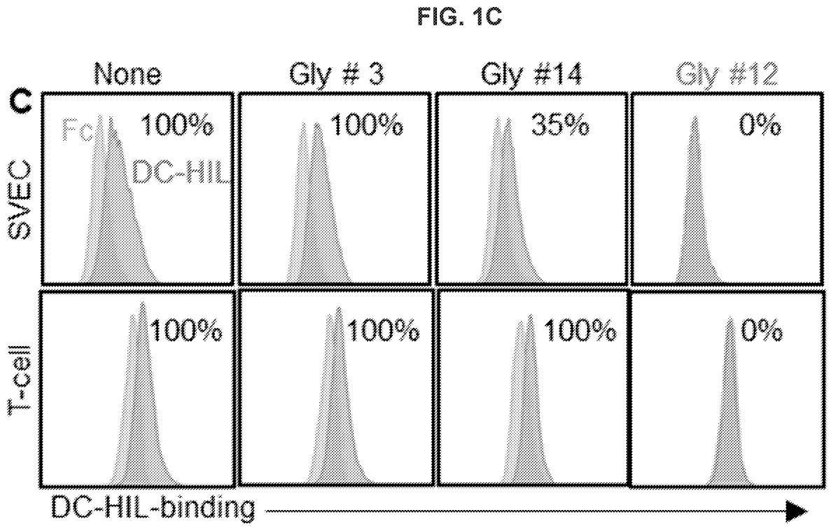

Various HS oligosaccharides (4-8-mers of HS disaccharide) were imprinted on microarray (Gycan Therapeutics Inc). These glycans were chosen based on sulfation patterns in mammalian cells (FIG. 1A). DC-HIL-Fc (10 mg/ml) was applied and then hybridized to the microaway and visualized by Cy-3 (mean±SD, n=6). FIG. 1B shows binding of DC-HIL-Fc to each glycan as average RFU on each glycan. SVEC mouse endothelial cells or activated T-cells were then incubated with DC-HIL-Fc (10 mg/ml) plus indicated oligoglycans, and DC-HIL binding was fluorescently stained with anti-Fc Ab and FACS analysis. FIG. 1C provides histograms showing binding of Fc control (blue) or DC-HIL-Fc (red), respectively. Based on the results in FIG. 1, Glycan #12 (GlcNS6S-GlcA-GlcNS6S-IdoA2S-GlcNS6S-IdoA2S-GlcNS6S-GlcA) was chosen for further experiments as it showed the strongest binding to DC-HiL.

### Example 2—Isolating sdAbs Specific for rHS by Screening Phage Display Library

Human antibody fragments were screened and selected according to published protocols (see e.g., Lee, C. M et al. (2007) “Selection of human antibody fragments by phage display”. Nat. Protoc., 2(11):3001-3008). A standard protocol provided by Source BioScience (Christ, D. “Selection of human antibody fragments by phage display”), a copy of which is incorporated herein as Appendix A, was followed. Briefly, a family of sdAbs generated from a single human VH framework (V3-23/D47) with diversity introduced in the antigen binding site. The repertoires were designed to contain short CDR3 of the heavy chains, while still maintaining good antigen binding properties. Diversified side chains were found in CDR1, CDR2, and CDR3: H27-33, H35, H50, H52-H54, H94, H95-H100(a-k), H101, H102.

The generated sdAb library was screened for selectivity toward the target antigen (glycan #12) using the following protocol. Briefly, 1×10¹¹ phages were pre-absorbed with 2 mg/ml Glycan #3 (See FIG. 1A) for 1 h. Unbound phages were recovered and plated on microwells precoated with 2 mg/ml Glycan #12. Bound phages were collected and amplified in TG1 E. coli. This panning was repeated 4-5 times to enrich clones specific for rHS. After the last panning, phage clone was individually tested for specificity to rHS. Positive clones were determined for DNA sequences and the uniqueness was identified by comparing with SdAb or Ig clones that are already registered in protein database (provided by NCI).

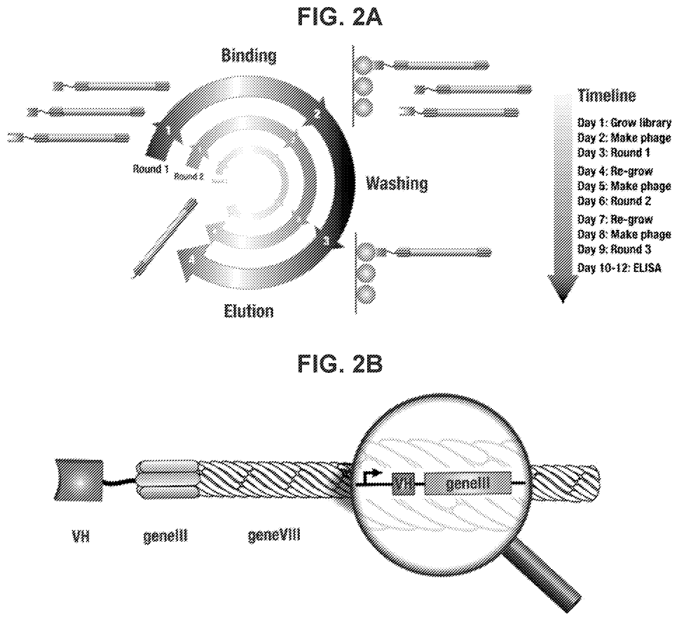

A more detailed overview of the phage selection process can be seen in FIG. 2A. As a first step of the protocol, the frozen glycerol stock of the repertoire phagemid in TG1 bacteria was thawed and phage produced in liquid culture after infection with KM13 helper phage (Steps 1-3). After phage production, 5×10¹² phages are incubated with antigen, thereby oversampling the antibody repertoires in excess of 1000-fold (Step 5). For the immobilization of antigen nonspecific adsorption to plastic is described in this protocol, although chemical coupling (by biotinylation) can also be utilized (Box 2). After incubation with antigen and stringent washes, phages are eluted by incubation with trypsin protease (Steps 5-6). This step will elute phage by cutting the c-myc tag between antibody fragment and the phage geneIII protein. It will also remove background infectivity originating from geneIII protein of the trypsin sensitive KM13 helper phage (geneIII protein originating from the repertoire phagemid is trypsin-resistant). The eluted phage is then used to infect TG1 bacteria and titres are determined by plating of dilution series (Steps 7-8). For subsequent rounds of selections, colonies from the first round are scraped from agar plates and phages are produced in liquid culture, PEG purified and selected by binding to antigen (Step 9). After three rounds of selection, individual clones are isolated, grown overnight (Step 10) and soluble fragments are produced in a 96-well format (Step 11-12). Finally, antigen-specific clones are identified by ELISA using a biotintylated anti-myc monoclonal in combination with streptavin-HRP and a colorimetric assay system (Step 13). Alternatively, antigen specific clones can be identified on phage by ELISA using an anti-phage conjugate (14-15). FIG. 2B shows an antibody fragment (e.g., a variable heavy chain, VH, in red) displayed as a fusion with the terminal phage geneIII protein (green). In accordance with the methods of this disclosure, both proteins were encoded by the phagmid DNA (magnified) and expressed from a common promoter. Also shown is the major gene VIII coat protein (in white).

### Example 3—Characterization of Anti-rHS SdAb

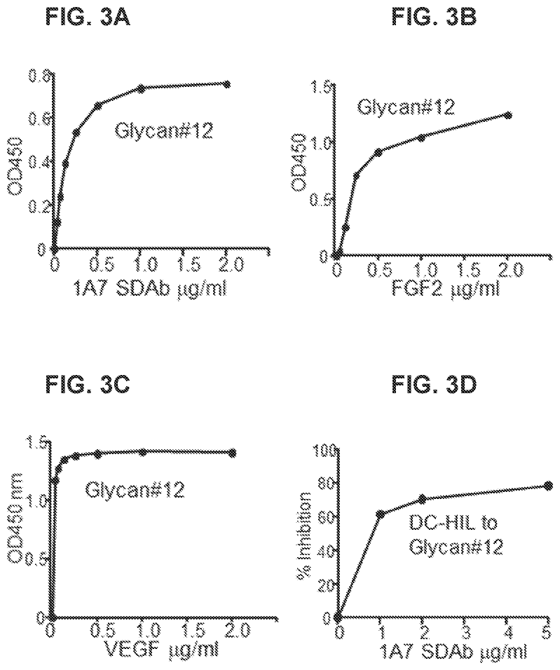

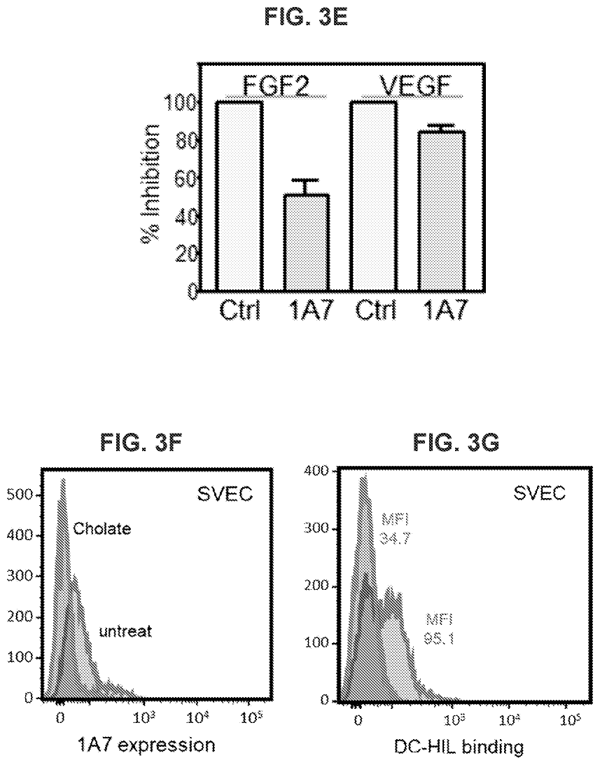

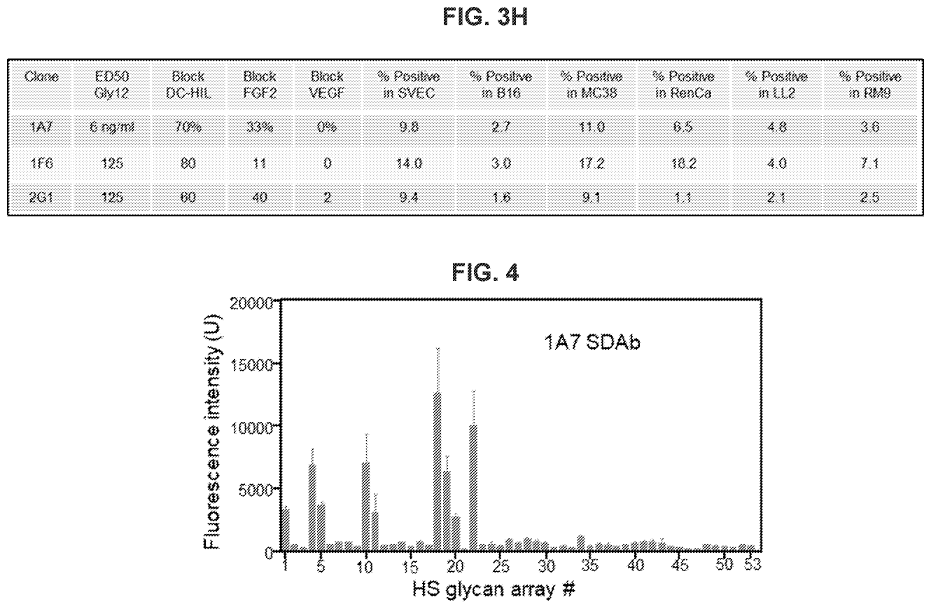

SdAbs were obtained as described in Example 2. All SdAb clones were purified from DNA-transfected CHO cells. FIGS. 2A, 2C and 2D shows the results of a binding assay between a representative SdAb clone (1A7 SdAb) for Glycan #12 as compared to binding of a non-ligand (FGF2) and a native ligand (VEGF). Biotinylated Glycan #12 (0.5 mg/ml) was immobilized to ELISA wells precoated with streptavidin (1 mg/ml) and incubated with increasing doses of 1A7 SdAb (FIG. 3A), FGF2 (negative control, FIG. 3B) and VEGF (positive control, FIG. 3C). After incubating for 1 h, wells were washed and binding measured by anti-c-myc Ab (OD₄₅₀); SdAb has the C-terminal c-myc tag. In FIG. 3D, DC-HIL-Fc (1 mg/ml) was incubated with Glycan #12-coated wells, with increasing dose of 1A7 SdAb. % inhibition is shown. FIG. 3D shows that 1A7 and DC-HIL-Fc compete for a similar binding site on Glycan #12. In FIG. 3E: FGF2 or VEGF (0.25 or 0.5 mg/ml) was added to Glycan #12-wells with 0.5 mg/ml of 1A7 or control SdAb (Ctrl), which is specific for KLH. % inhibition is shown. To examine the expression of the 1A7 epitope, immortalized murine epithelial cells (SVEC) were treated with 50 mM Sodium Cholate (red histogram) or untreated (Blue). Next day, cells were analyzed by FACS for expression of 1A7 epitope (FIG. 3F). FIG. 3G shows DC-HIL-binding on SVEC after pretreatment with 1A7 or control (red and blue histograms, respectively). FIG. 3H summarizes all data from experiments of binding/inhibition and expression analysis on different tumor lines. “Block DC-HIL (or VEGF or FGF2)” means blocking of binding to Glycan #12.

### Example 4—HS Glycan Microarray Analysis of 1A7 SdAb

1A7 SdAb (10 μg) was hybridized with HS glycan microarray consisting of 53 glycan compounds (Glycan Therapeutics Inc) and fluorescently labeled with anti-c-myc. Hybridized arrays were analyzed for fluorescence intensity (U); average±sd, n=100). SdAb has the C-terminal c-myc tag. Data is shown in FIG. 4. The figure shows that SdAb 1A7 shows a robust affinity for Glycan #12, along with other HS glycans.

### Example 5—Kinetics of rHS Expression after T Cell Activation

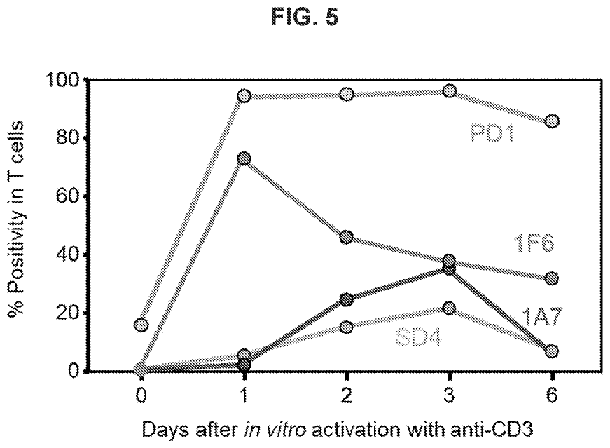

Pan-T cells were isolated from peripheral blood of a healthy donor and incubated in well (2×10⁵ cells/well) with CD2/CD3/CD28-anti-biotin MACS Beads (cell: bead ratio is 1:1.5). At indicated time point, cells were harvested and examined by FACS for expression of 1A7, 1F6 epitope, Syndecan-4 (SD4) or PD-1. Expression is shown by % positive cells among total T cells. FIG. 5 indicates that T-cell activation induces expression of both 1A7 and 1F6 epitopes but 1F6 epitope expression precedes 1A7 expression.

### Example 6—Immuno-Histochemistry (IHC) Staining of Mouse Normal Lung with Anti-rHS SdAb

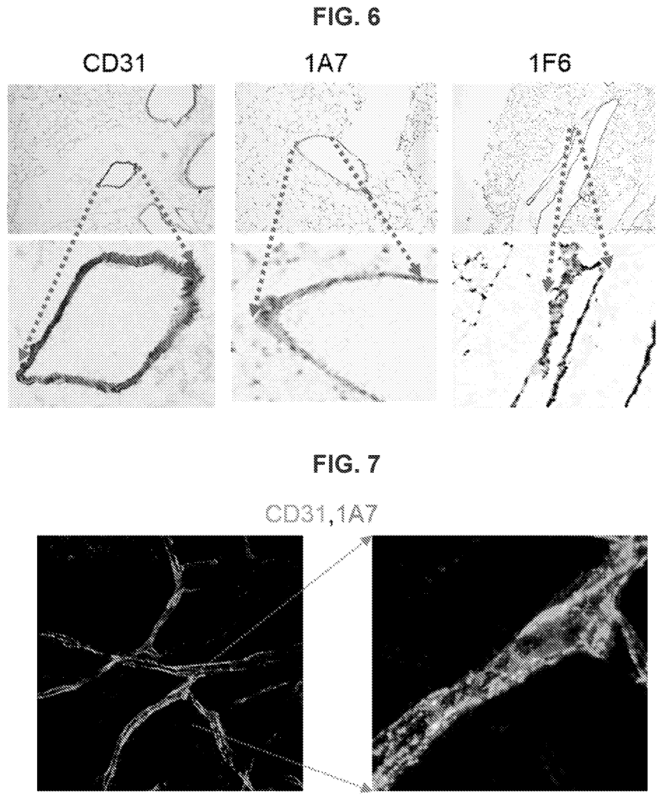

To determine the expression of the target glycan (Glycan #12) in vivo, an immunohistochemistry staining of normal mouse lung was performed. Whole lungs of normal mice were perfused with PBS and fixed. Tissue sections were stained with anti-CD31, 1A7 or 1F6 SdAb (0.5 mg/ml or 10 mg/ml, respectively). ImmPACT Vector Red Substrate was used for color development and followed by counterstaining with hematoxylin. All endothelial cells were stained for CD31, but only some for 1A7 or 1F6 indicating a restricted expression of target glycan (FIG. 6).

### Example 7—Whole Mount Staining of Ear Skin with 1A7 and CD31 Ab

Another experiment to determine expression of the target glycan (Glycan #12) in vivo was performed. Whole ear of untreated mice was excised, fixed, and stained with 1A7 SdAb (shown in red fluorescence) and anti-CD31 Ab (in green, 10 mg/ml and 0.5 mh/ml, respectively), and subjected to confocal microscopy. FIG. 7 shows that 1A7-stains are confined to certain areas in blood vessels. The yellow window is enlarged and shown at the right.

### Example 8—Expression of rHS in Normal Human Lung

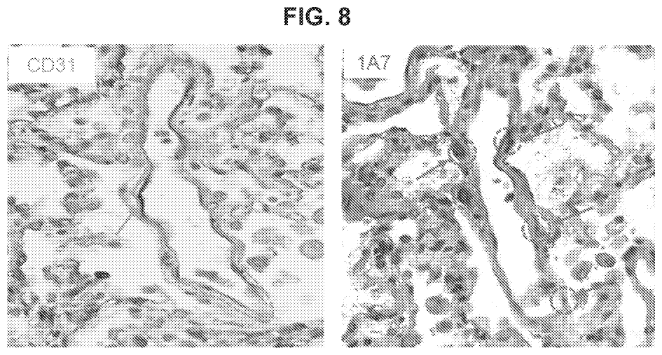

An experiment to determine the expression of the target glycan (Glycan #12) in human tissue was performed. Normal human lung specimens (US Biomax, Inc) were stained with anti-CD31 or 1A7 Ab (0.5 mg/ml or 10 mg/ml, respectively). DAB purple substrate was used for color development, followed by hematoxylin counterstaining. A similar staining pattern to mouse (e.g., FIG. 6) was observed. CD31 stains are located on the apical side of endothelial cells, but 1A7 stains at the basal side (FIG. 8). It is possible that 1A7 SDAb are immuno-reactive to mouse and human because heparan sulfate structures are almost the same among mammals.

### Example 9—Effect of rHS (Glycan #12) on DC-HIL-Induced Angiogenesis

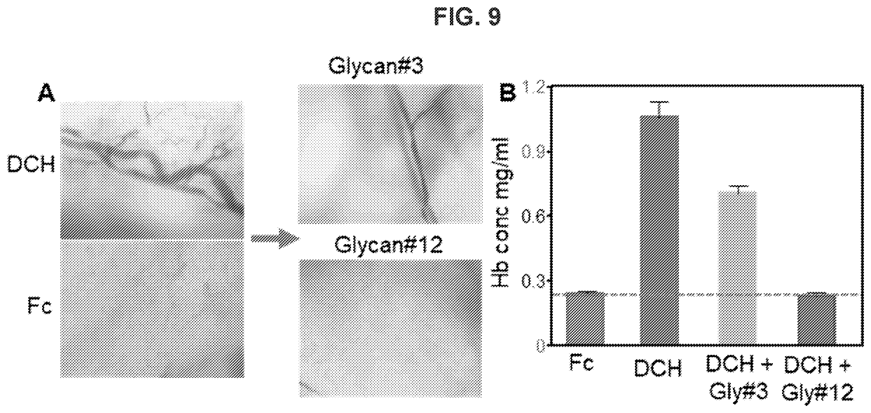

In order to test the role rHS (Glycan #12) plays in controlling angiogenesis, DC-HIL-Fc (DCH) or Fc (200 nM) was mixed with 10× molecular excess of Glycan #3 or Glycan #12 (See FIG. 1A) and added to matrigel, and implanted s.c. into mice; 2 weeks later, plugs were excised and observed for blood vessels under microscope. Representative photos are shown (FIG. 9A). Plugs were quantified for Hb content (FIG. 9B). Red-dashed line indicates Hb level in Fc-control plug. Data are shown average±SD, n=4. Collectively, FIGS. 9A and 9B shows that rHS (Glycan #12) completely abrogated DC-HIL-induced angiogenesis.

### Example 10—Effect of rHS (Glycan #12) on Tumor Growth and Expression of Angiogenic Factors

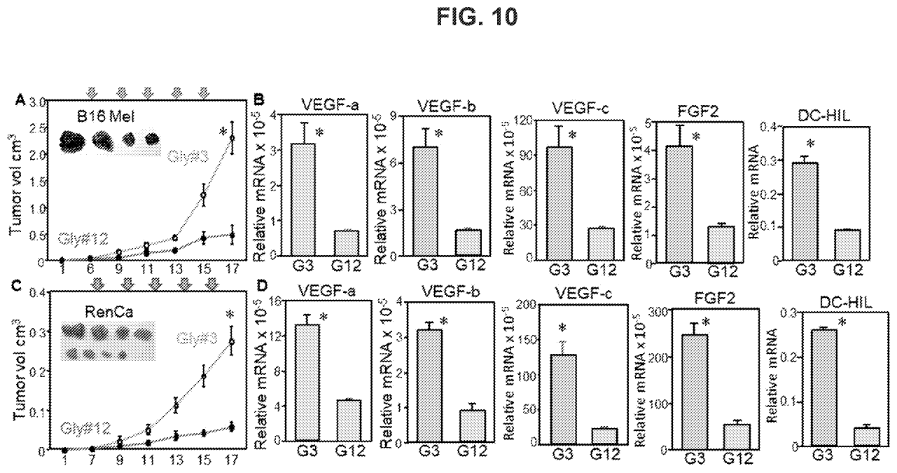

B16 melanoma cells (5×10⁵ cells/mouse) were implanted s.c. into B6 mice (n=4); on day 6, 1 mg of Glycan #3 (G3) or Glycan #12 (G12, see FIG. 1A) was injected intra-tumorally 5 times every 2 days. Tumor volume was measured (FIG. 10A). On day 17, tumors (photos of 2 representatives) were excised. FIG. 10B. shows total RNA prepared and quantified by qRT-PCR for expression of angiogenic factors and shown as relative to GAPDH; data±SD, n=4. Similar experiments were performed with RenCa kidney cancer in BALB/c mice and data are shown in FIG. 10C (tumor volume) and 10D (expression of angiogenic factors). Data shown is representative of 2 experiments. *p<0.0001. Collectively, FIGS. 10A-10D show that injection of Glycan #12 markedly attenuated growth of melanoma and kidney cancer, and inhibited tumor expression of angiogenic factors.

### Example 11—Effect of 1A7 Ab Single Treatment on Tumor Growth and Lung Metastasis

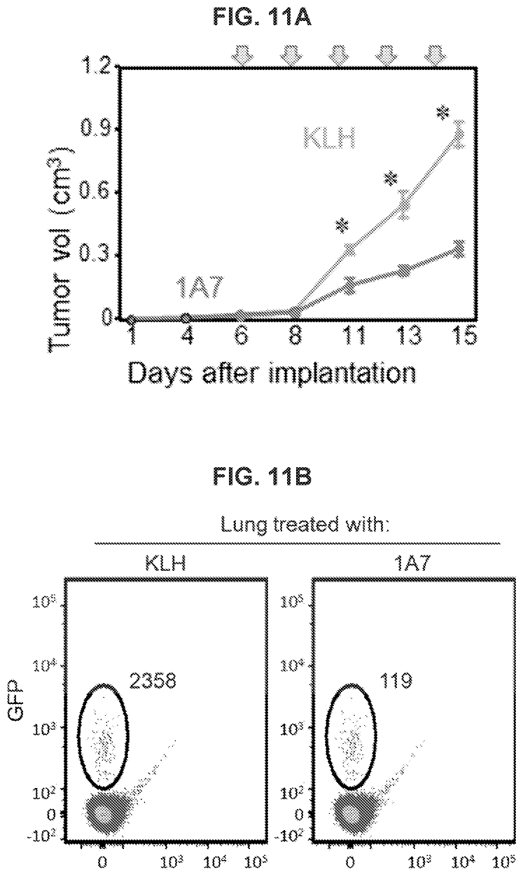

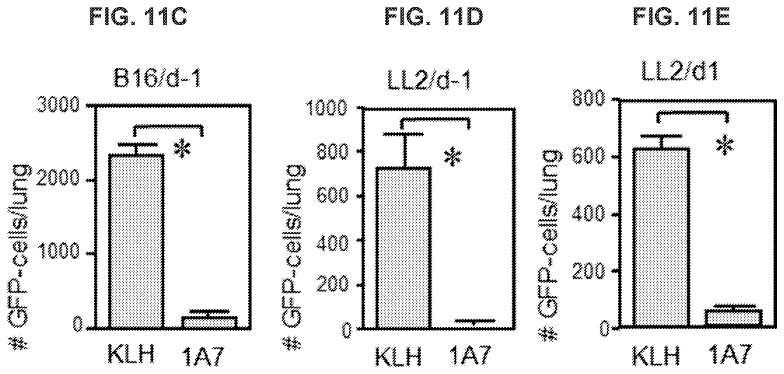

On day 6 after implantation s.c. of B16 melanoma cells (2×10⁵ cells/mouse), mice were treated i.p. with 1A7 or KLH Ab (10 mg/mouse, every 2 days, 5×, shown by reverse arrows). Tumor volume was measured and shown in FIG. 11A (median±sd, n=5). In separate experiments, tumor free mice were injected intravenously (i.v) with 1A7 or KLH (10 mg/mouse) either one day before (d-1) or one day after (d1) intravenous (i.v) injection with B16 or LL2 cells (2×10⁵ cells/mouse, n=5). On day 12 (for mice injected with B16 cells) or day 18 (for mice injected with LL2 cells), the number of GFP+ cells in 5×10⁵ lung cells was determined via FACS. Representative FACS data is shown in FIG. 11B. FIG. 11C-11E summarize numbers of positive cells in each condition. FIG. 10C shows cells in mice injected with B16 cells a day after treatment with KLH or 1A7. FIG. 11D shows cells in mice injected with LL2 cells a day after treatment with KLH or 1A7. FIG. 11E shows positive cells in mice injected with LL2 cells and then treated a day later (d1) with KLH or 1A7. In each instance, administration of a single treatment of the 1A7 antibody significantly reduced tumor growth (FIG. 11A) and lung metastasis (FIG. 11B-E).

### Example 12—Comparison of 1A7 SdAb Inhibition of B16 Metastasis with Other Treatments

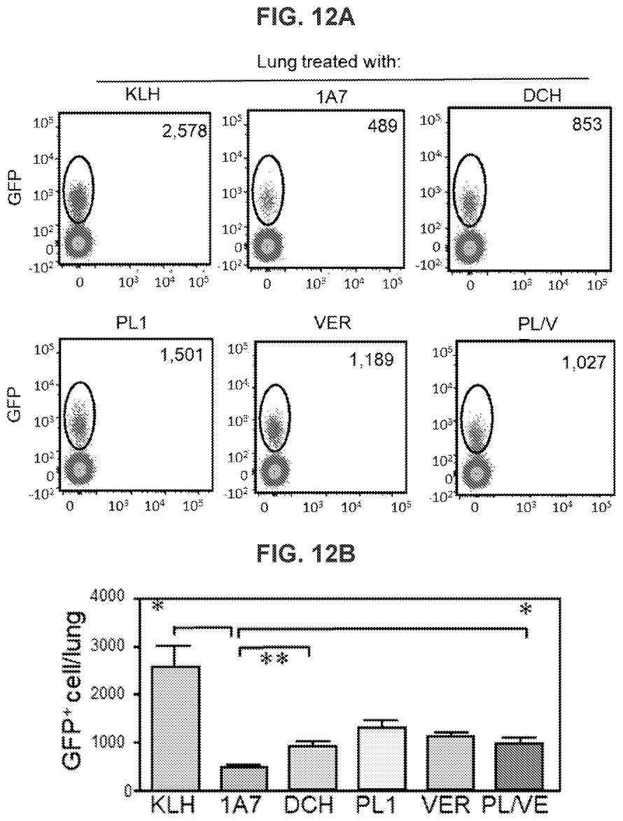

1A7 SdAb was compared to other standard treatments (e.g., anti-PDL1 antibody, anti-VEGFR2 antibodies) for its ability to inhibit metastasis using the B16 cell model. Mice (n=5) were injected i.v. with KLH, 1A7 SdAb (10 mg/mouse), anti-DC-HIL (DCH), anti-PDL1 (PL1), anti-VEGFR2 (VER) or combined PL1/VER (PL/VE) mAb (200 mg/mouse). Next day, mice were given i.v. injection of 2×10⁵ B16 cells/mouse. Note: Ab injection was a one-time treatment. On Day 14, lung was enumerated by FACS for GFP⁺ cells (FIG. 12A) and the number in 5×10⁵ lung cells (average±sd, n=5) are summarized (FIG. 12B). *p<0.0001; **p=0.007. Data are representative of 2 separate experiments. to mouse and human because heparan sulfate structures are almost same among mammalians. Collectively, FIGS. 12A and 12B show that 1A7 SdAb inhibited growth of B16 metastasis, more than currently FDA-approved drugs (anti-PDL1 and anti-VEGF Ab).

### Example 13—B16 Cell-Derived Soluble Factors Upregulated 1A7 Epitope and HSST Genes

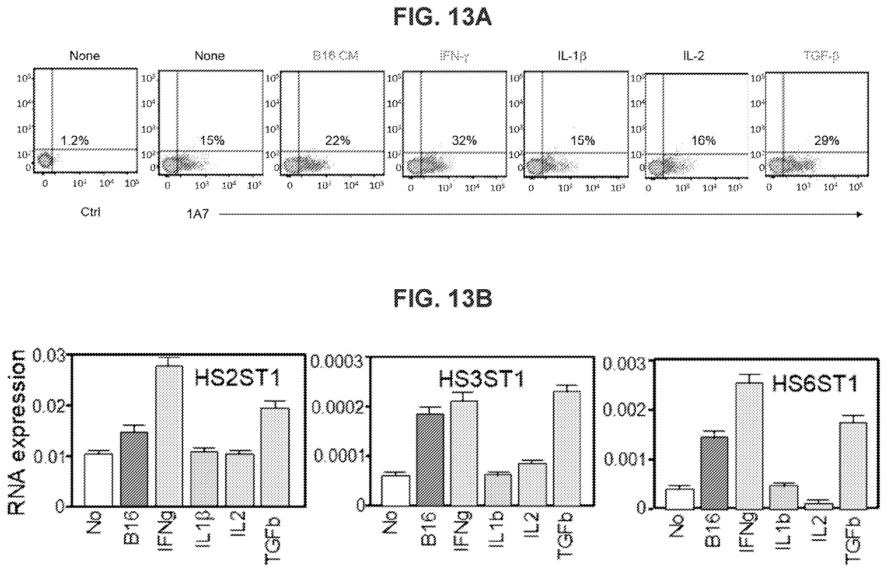

An experiment was performed to determine whether 1A7 epitope or heparan sulfate sulfo-transferase (HSST) genes were affected by B16-derived soluble factors. SVEC were cultured with 20% of conditioned media (CM) of B16 melanoma cell culture or selected cytokines (IFN-γ, IL-1β, IL-2, and TGF-β) (10 ng/ml) or media (None). The next day, cells were harvested and examined by FACS for expression of 1A7 epitope or control (Ctrl) (FIG. 13A) and by qRT-PCR for RNA expression of HS2ST1, HS3ST1, and HS6ST1 genes relative to GAPDH (FIG. 13B). It was found that B16 cell-derived soluble factors upregulated 1A7 epitope and HSST genes.

### Example 14 Effect of 1A7 Anti-rHS on T Cell Suppression

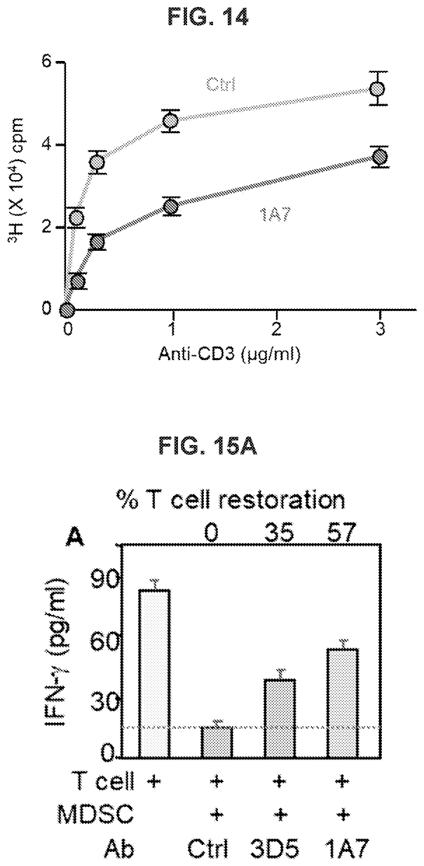

The ability of 1A7 to affect induction of T-cell activation using anti-CD3 antibodies was tested. Pan-T cells isolated from blood sample of a healthy donor were incubated with wells (2×10⁵ cells/well) pre-coated with 1A7 or KLH control Ab (4 mg/well) plus increasing doses of anti-CD3 Ab. After incubation for 2 days, ³H-thymidine was pulsed for 16 h, harvested, and counted for cpm in cells. FIG. 14 shows average ³H thymidine counts recorded for increasing concentrations of 1A7. Cells treated with 1A7 showed significantly less replication (activation). Data are shown average±SD, n=3

### Example 15 Combined Treatment with Anti-rHS and Anti-DC-HIL mAb Markedly Reversed MDSC Function

Monocytic myeloid derived suppressor cells (MDSCs) actively suppress the immune system in many cancers by suppressing expression of IFN-γ. An experiment was performed to test the effect of anti-rHS antibodies and anti-DC-HIL antibodies on MDSC induced suppression of T-cell function. T-cells and monocytic MDSC isolated from blood of patients with colorectal cancer (FIG. 15A) or renal cell carcinoma (FIG. 15B) were co-cultured (1:1 cell ratio) with anti-CD3/28 Ab plus control (Ctrl) or 3D5 anti-DC-HIL Ab (50 mg/ml) or 1A7 anti-rHS Ab (10 mg/ml) or a combination of the last 2 Ab (3D5 and 1A7). IFN-γ was assayed 5 days later (median±sd, 3 wells). Results are expressed as % T-cell restoration, with red-dashed lines corresponding to level of IFN-g suppression by MDSC. MDSC from patients in FIGS. 15A and 15B showed DC-HIL at 60% and 90% of total MDSC, respectively. The data collectively shows that combined treatment with anti-rHS and anti-DC-HIL mAb markedly reverses MDSC induced suppression of T-cell function, restoring IFN-γ expression.

### Example 16—Single Injection of 1A7 SdAb Inhibited Lung Metastasis of Human Melanoma Cells in Xenograft Mice

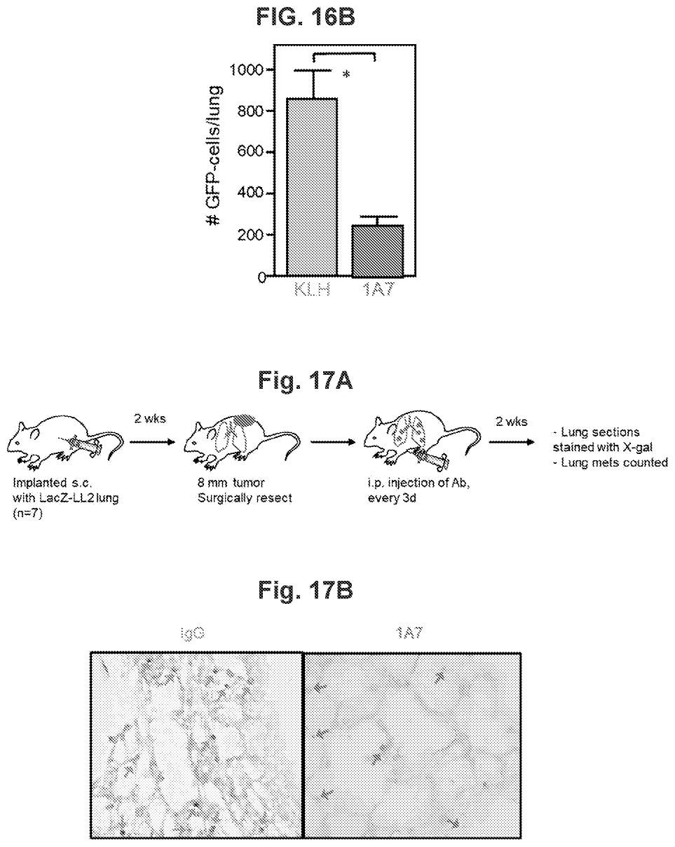

A final experiment was performed to determine the effect of an anti-rHS antibody on human lung metastasis in a xenograft mouse model. Immuno-deficient NSG mice (n=5) were injected i.v. with KLH or 1A7 SdAb (10 mg/mouse). Next day, mice were given i.v. injection of 2×10⁵ human SK-MEL-28 melanoma cell line. Note: Ab injection is just one time. On Day 14, lung was enumerated by FACS for GFP⁺ cells (FIG. 16A) and the number in 5×10⁵ lung cells (average±sd, n=5) are summarized (FIG. 16B). *p<0.0001. Data are representative of 2 separate experiments. 1A7 single injection decreased human melanoma in lung by 84% (FIGS. 16A and 16B).

### Example 17-1A7 Treatment Significantly Reduced Spontaneous Lung Metastasis from the Primary Tumor In Vivo

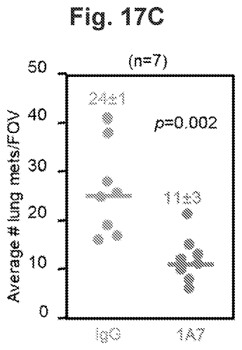

In order to treat spontaneous metastasis in vivo with the rHS specific sdABs described herein, a protocol shown in FIG. 17A was performed. Briefly, mice were implanted s.c. with LacZ-LL2 cells, 2 weeks later, tumor was resected surgically. Mice were then treated with Ab injection every 3 days. 2 weeks later, lungs were excised and stained with X-gal (labeled LacZ-LL2 cells in blue) and blue stains were counted. FIG. 17B provides representative images of lung sections stained with X-gal, showing by green or red arrows. The number of tumor cells per field of view (FOV) were counted and plotted in FIG. 17C alongside and average of 5 FOVs. Each dot represents an individual mouse. As shown in FIG. 17C, 1A7 treatment significantly reduced spontaneous lung metastasis from the primary tumor in vivo.

### Example 18—Blood sDC-HIL Levels in Tumor Response of Metastatic Renal Cell Carcinoma (mRCC) Patients

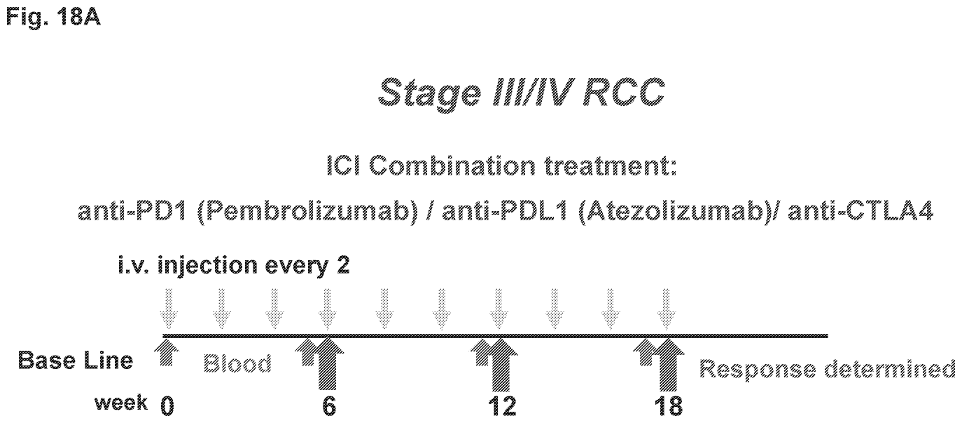

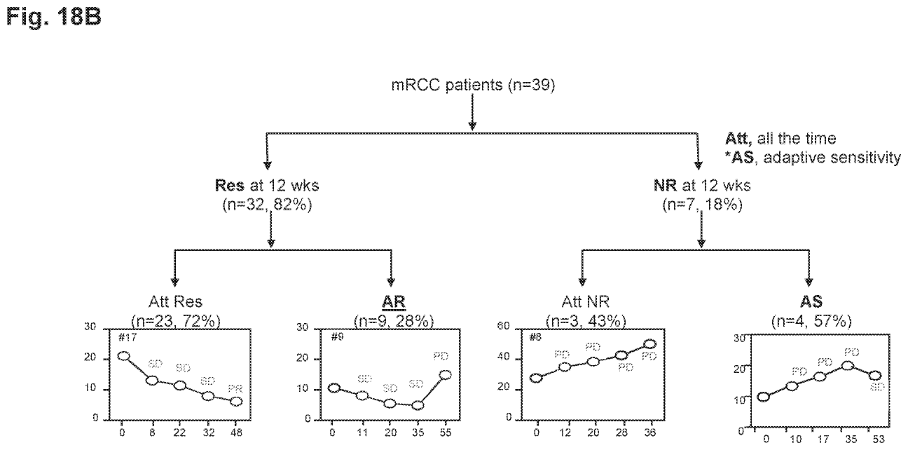

Stage III/IV RCC patients (n=39) were treated with immune checkpoint inhibitors (ICIs) and evaluated for tumor response at every follow-up (weeks post-treatment). Plasma of the patients was collected at 0 (base line), 6, 12, 18 weeks (FIG. 18A). Tumor response was assessed by Response Evaluation Criteria in Solid Tumours (RECIST); progressive disease (PD), stable disease (SD), partial response (PR) and complete response (CR). At 12 weeks follow-up, patients were categorized into responders (Res, who displayed SD, PR or CR) and non-responders (NR, SD). Initial responders were sorted into two phenotypes; all the time responders (Att Res) who always displayed response to ICIs and acquired resistance (AR) who became unresponsive to ICIs. Non-responders also diverged into two phenotypes; all the time non-responders (Att NR) who never exhibited response and acquired sensitivity (AS) who acquired response to ICIs. Blood samples were collected from patients at every follow-up visit and determined by ELISA for soluble DC-HIL (sDC-HIL) protein. Changes over time in sDC-HIL (ng/ml) are plotted with tumor response of representative patients of each cohort (FIG. 18B). Att Res cohort expressed gradually decreased sDC-HIL level and remained low levels all the time. AR cohort displayed good response for a while, but suddenly they were unresponsive. In this transition, sDC-HIL level rose quickly. Att NR cohort kept sDC-HIL levels very high. AS cohort displayed poor response with high sDC-HIL levels, but suddenly showed good response. In this transition, sDC-HIL levels went down.

### Example 19—Kinetics of sDC-HIL in Individual Patient Post-ICI Therapy

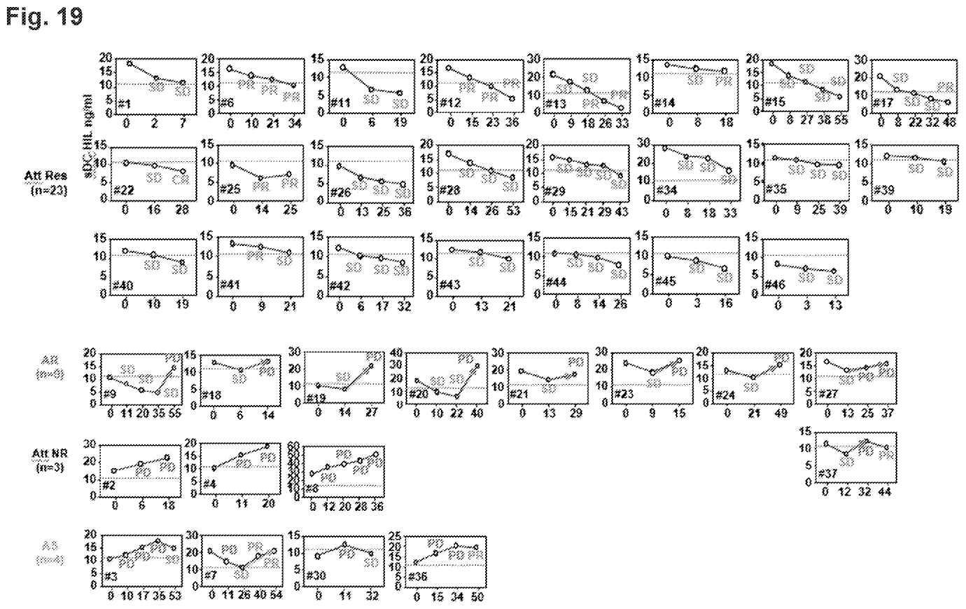

The same follow-up studies as in Example 18 were performed with 39 patients and individually chronological changes in sDC-HIL levels and tumor response are shown in FIG. 19 (red dotted lines show the average sDC-HIL levels among initial responders). All patients examined showed significant association of high sDC-HIL levels and poor response, except one patient (P #7) with AS phenotype that showed opposite relationship: high sDC-HIL levels, but significant response. These data indicate that blood sDC-HIL is a promising biomarker for poor or resistance to ICI therapy.

### Example 20—rHS Ab Treatment Inhibited Tumor Growth of B16 Mouse Melanoma More than PD-1 Ab

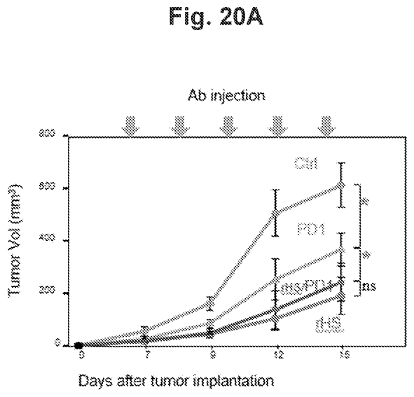

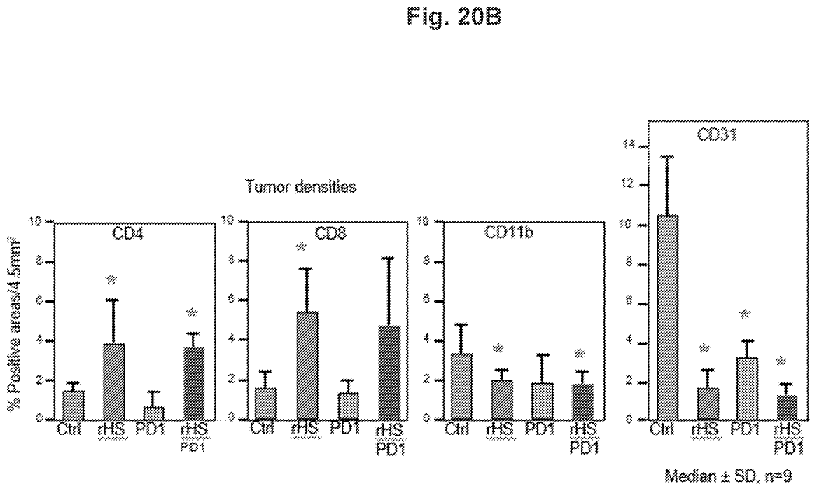

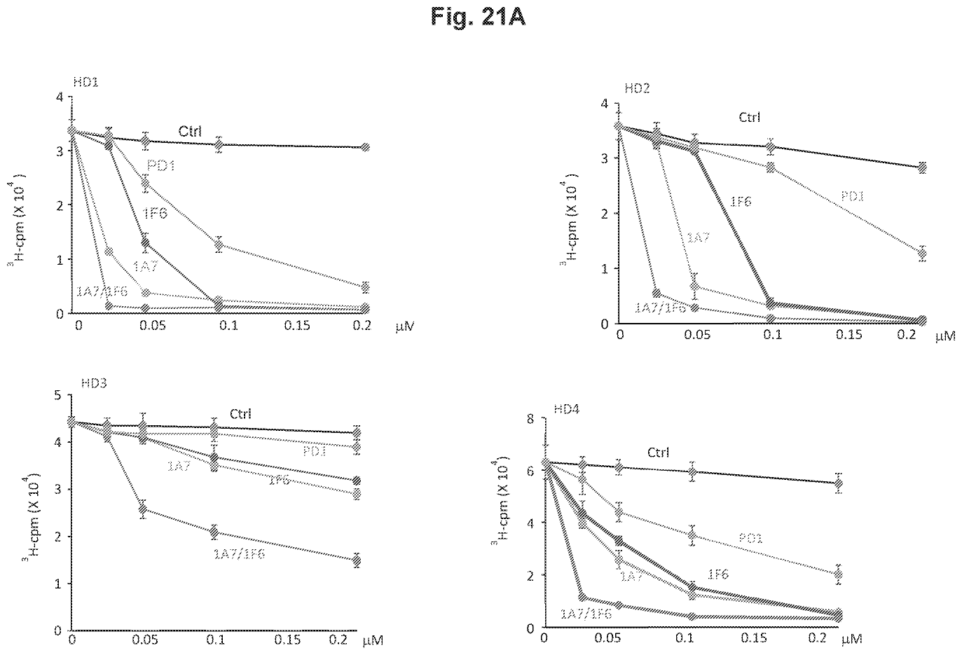

Antitumor activity of rHS Ab to B16 melanoma was examined. Briefly, B16F10 melanoma cells (5×105 cells/mouse) were implanted s.c. into B57BL/6 mice (n=5). Six days later, mice were treated with i.p. injection of Ab every 2 days; 1A7 rHS Ab (20 mg/mouse), PD1 (200 mg/mouse), rHS/PD1 (20 mg and 200 mg, respectively) or control (Ctrl) Ab (200 mg/mouse). Tumor volume was measured every 2 days (FIG. 20A). On day 15, all mice were euthanized, tumors were excised and IHC-stained with marker Ab (CD4, CD8, CD11b, and CD31) (FIG. 20B). Positive staining is assayed by % positively stained areas in 4.5 mm2 view (mean±sd, n=5). These data show that rHS Ab produced better outcomes than PD1 Ab, accompanied with increased tumor densities of CD4 and CD8 T cells but decreased densities of CD11 b and microvacuoles. Thus, rHS Ab inhibited melanoma growth, reversing immune suppression and tumor angiogenesis. Example 21—Stimulation of rHS-bearing receptors on human T cells by rHS Ab attenuates CD3-proliferation response significantly more than PD1 receptor by specific Ab.

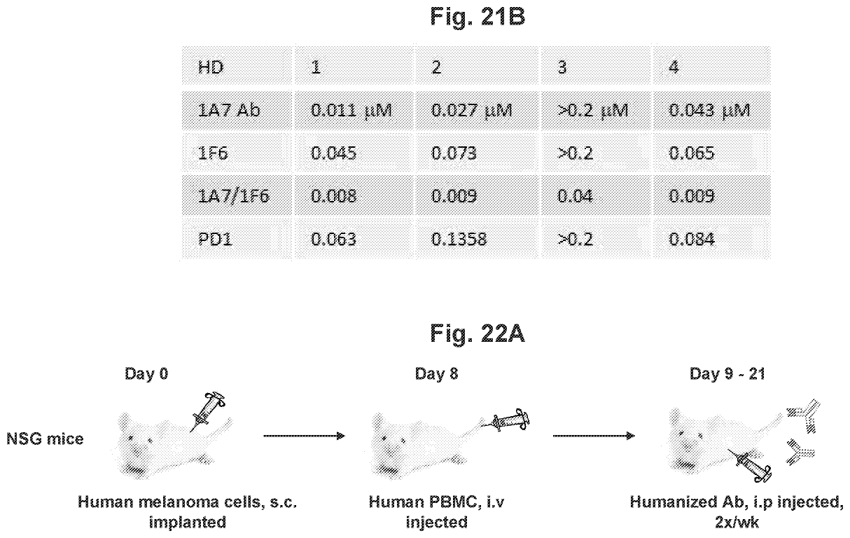

CD3 T cells isolated from peripheral blood of healthy donors (HD, 4 individuals) were cultured with anti-CD3 Ab (a constant dose of 0.3 mg/ml) and increasing doses (mM) of test Ab immobilized onto 96-plate microwells. Ab are control (Ctrl) IgG, PD1 Ab (Keytruda, pembrolizumab), 1A7 and 1F6 rHS single domain Ab. After culturing for 2 days, proliferation was measured by 3H-thymidine incorporation (cpm) into T cells (FIG. 21A). Each Ab was assessed for the dose (mM) required for 50% inhibition of 3H-cpm in control culture with no test Ab; IC50 value (FIG. 21B). These results documented that rHS-receptors are potent inhibitors of T cell activation at least 10-fold greater than PD1 receptor.

### Example 22—Comparison of Anti-DC-HIL/rHS Ab Vs. PD1 Keytruda in Efficacy to Treat Human Melanoma SK-MEL-28 in PBMC-Humanized Mice

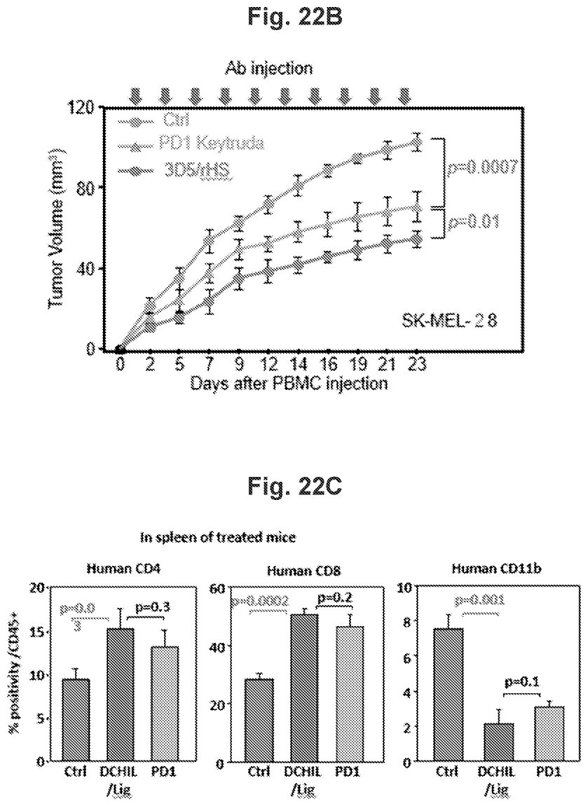

Experimental design to evaluate efficacy of test Ab to inhibit tumor growth of human melanoma SK-MEL-28. Day 0, immunodeficient NSG mice (n=5) were injected s.c. with human melanoma cells (1×106 cells/mouse); Day 8, normal human PBMC (1×107 cells/mouse) were injected i.v.; and Day 9-21, mice were treated with i.p. injection of Ab twice a week. Control (Ctrl) Ab, PD1 Keytruda (both 200 mg/mouse) or 3D5/rHS (a mixture of 3D5 anti-DC-HIL mAb, 200 mg, and 1A7 rHS Ab, 40 mg per mouse) (FIG. 22A). Tumor volume was measured every 2 days (FIG. 22B). Starting at day 5 after injection, tumor growth gradually diverged between Keytruda and DC-HIL/Ligand blockers. At every time point, tumor size in DC-HIL/Ligand blocker-treated mice was always smaller than Keytruda. On day 23, all mice were euthanized, and spleens excised and determined by FACS for % leukocyte subpopulation among CD45+ human cells (FIG. 22C, two-way ANOVA statistical analysis was performed between groups. P values heighted in red and black fonts are significant and insignificant, respectively). DC-HIL Ligand blocker increased number of human T cells more than Keytruda. By contrast, CD11 b myeloid cells, having suppressor activity, were decreased. These data indicate that combined 3D5 Ab/rHS Ab treatment inhibited growth of human melanoma line SK-MEL-28 more than PD1 Keytruda, in humanized mice.

### Example 23—Treatment of Human Melanoma SK-MEL-5 by Anti-DC-HIL/rHS Ab Vs. PD1 Keytruda in PBMC-Humanized Mice

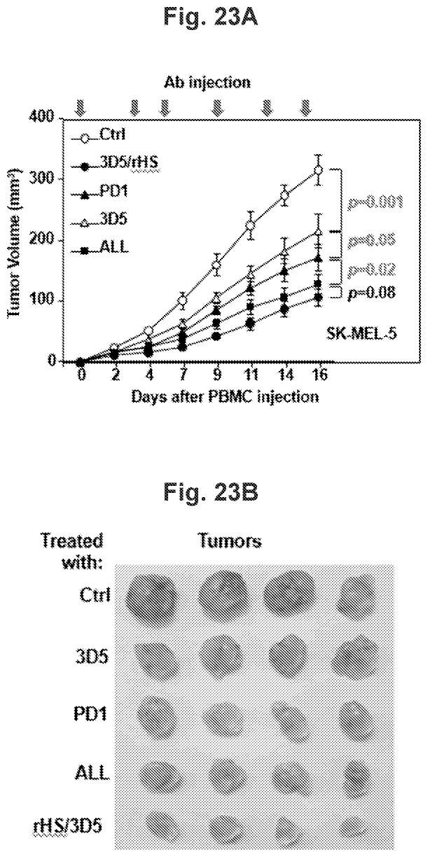

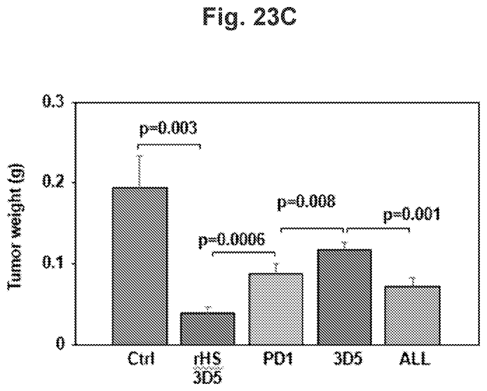

NSG mice (n=5) were similarly treated with human PBMC and Ab as in Example 22, but with another human melanoma line, SK-MEL-5. Tumor growth curves of SK-MEL-5 on mice treated with control Ab, 3D5, PD1 Keytruda, 3D5/rHS, or a mixture of all Ab are shown in FIG. 23A (Ab doses were same as Example 22). Statistical analysis (p value) was performed between treatment groups (2-ways ANOVA). Tumors were excised on day 16 and photographed (FIG. 23B). The tumors were weighed (tumor weight (g) is summarized in FIG. 23C). These data indicate that combined 3D5 Ab/rHS Ab treatment inhibited growth of human melanoma line SK-MEL-5 more than PD1 Keytruda in humanized mice.

What is claimed is:

1. A single domain antibody (sdAb) comprising a variable heavy domain having a specific affinity for a heparan sulfate glycan having a structure of Formula I (GlcNS6S-GlcA-GlcNS6S-IdoA2S-GlcNS6S-IdoA2S-GlcNS6S-GlcA)

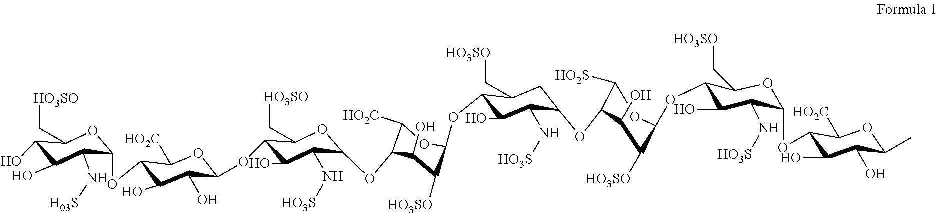

2. The single domain antibody of claim 1, wherein the sdAb comprises a heavy chain complementary determining region 1 (CDR_{H1}) having an amino acid sequence comprising GXKVXXXX, where each X is independently any amino acid (SEQ ID NO: 1).

3. The single domain antibody of claim 2, wherein the sdAb comprises a heavy chain complementary determining region 1 (CDR_{H1}) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2).

4. The single domain antibody of any one of claim 2 or 3, wherein the sdAb comprises a heavy chain complementary determining region 1 (CDR_{H1}) having an amino acid sequence having at least 60%, at least 70%, at least 80% or at least 90% sequence identity to GVKVSTKS (SEQ ID NO: 3) or GFKVTSQA (SEQ ID NO: 4).

5. The single domain antibody of any one of claims 2 to 4, wherein the sdAb comprises a heavy chain complementary determining region 1 (CDR_{H1}) comprising GVKVSTKS (SEQ ID NO: 3) or GFKVTSQA (SEQ ID NO: 4).

6. The single domain antibody of any one of claims 1 to 5, wherein the sdAb comprises a heavy chain complementary determining region 2 (CDR_{H2}) having an amino acid sequence comprising IXXXXGST, where each X is independently any amino acid (SEQ ID NO: 5).

7. The single domain antibody of claim 6, wherein the sdAb comprises a heavy chain complementary determining region 2 (CDR_{H2}) having an amino acid sequence comprising IXXXXGST, where each X is independently selected from the group consisting of A(Ala), R(Arg), K(Lys), N(Agn), G(Gly), and D (Asp) (SEQ ID NO: 6).

8. The single domain antibody of any one of claim 6 or 7, wherein the sdAb comprises a heavy chain complementary determining region 2 (CDR_{H2}) having an amino acid sequence having at least 60%, at least 70%, at least 80% or at least 90% sequence identity to IARNDGST (SEQ ID NO: 7) or IRKGNGST (SEQ ID NO: 8).

9. The single domain antibody of any one of claims 6 to 8, wherein the sdAb comprises a heavy chain complementary determining region 2 (CDR_{H2}) having an amino acid sequence comprising IARNDGST (SEQ ID NO: 7) or IRKGNGST (SEQ ID NO: 8).

10. The single domain antibody of any one of claims 1 to 9, wherein the sdAb comprises a heavy chain complementary determining region 3 (CDR_{H3}) having an amino acid sequence comprising A-X1-X2-K-X1-X1-X1-X1-S-X2-X2-Q-X2-X2-X1-X1-S(SEQ ID NO: 9) wherein each X1 is independently any amino acid and each X2 is independently absent or is any amino acid.

11. The single domain antibody of claim 10, wherein the sdAb comprises a heavy chain complementary determining region 3 (CDR_{H3}) having an amino acid sequence comprising A-X1-X2-K-X1-X1-X1-X1-S-X2-X2-Q-X2-X2-X1-X1-S(SEQ ID NO: 10) wherein each X1 is selected from the group consisting of R (Arg), T(Thr), K(Lys), A(Ala), N(Agn), Y(Tyr), G(Gly), I(Ile), L(Leu) and each X2 is either absent or is selected from the group consisting of M (Met), K(Lys) R(Arg), P(Pro), and H(His).

12. The single domain antibody of any one of claim 10 or 11, wherein the sdAb comprises a heavy chain complementary determining region 3 (CDR_{H3}) having an amino acid sequence having at least 60%, at least 70%, at least 80% or at least 90% sequence identity to ARMKKNGGSKRQPHIRS (SEQ ID NO: 16) or ATKAYIKSQLGS (SEQ ID NO: 17).

13. The single domain antibody of any one of claims 10 to 12, wherein the sdAb comprises a heavy chain complementary determining region 3 (CDR_{H3}) having an amino acid sequence comprising ARMKKNGGSKRQPHIRS (SEQ ID NO: 16) or ATKAYIKSQLGS (SEQ ID NO: 17).

14. The single domain antibody of any one of claims 1 to 13, wherein the sdAb comprises:
(a) a heavy chain complementary determining region 1 (CDR_{H1}) having an amino acid sequence comprising GXKVXXXX, where each X is independently any amino acid (SEQ ID NO: 1);
(b) a heavy chain complementary determining region 2 (CDR_{H2}) having an amino acid sequence comprising IXXXXGST, where each X is independently any amino acid (SEQ ID NO: 5); and
(c) a heavy chain complementary determining region 3 (CDR_{H3}) having an amino acid sequence comprising A-X1-X2-K-X1-X1-X1-X1-S-X2-X2-Q-X2-X2-X1-X1-S(SEQ ID NO: 9) wherein each X1 is independently any amino acid and each X2 is independently absent or is any amino acid.

15. The single domain antibody of any one of claims 1 to 14, wherein the sdAb comprises:
(a) a heavy chain complementary determining region 1 (CDR_{H1}) having an amino acid sequence comprising GXKVXXXX, where each X is independently selected from the group consisting of V(Val), F(Phe), S(Ser), T(Tyr), K(Lys), Q(Gln), and A(Ala) (SEQ ID NO: 2);
(b) a heavy chain complementary determining region 2 (CDR_{H2}) having an amino acid sequence comprising IXXXXGST, where each X is independently selected from the group consisting of A(Ala), R(Arg), K(Lys), N(Agn), G(Gly), and D (Asp) (SEQ ID NO: 6); and
(c) a heavy chain complementary determining region 3 (CDR_{H3}) having an amino acid sequence comprising A-X1-X2-K-X1-X1-X1-X1-S-X2-X2-Q-X2-X2-X1-X1-S(SEQ ID NO: 9) wherein each X1 is selected from the group consisting of R (Arg), T(Thr), K(Lys), A(Ala), N(Agn), Y(Tyr), G(Gly), I(Ile), L(Leu) and each X2 is either absent or is selected from the group consisting of M (Met), K(Lys) R(Arg), P(Pro), and H(His).

16. The single domain antibody of any one of claims 1 to 15, wherein the sdAb comprises:
(a) a heavy chain complementary determining region 1 (CDR_{H1}) having an amino acid sequence comprising GVKVSTKS (SEQ ID NO: 3) or GFKVTSQA (SEQ ID NO: 4);
(b) a heavy chain complementary determining region 2 (CDR_{H2}) having an amino acid sequence comprising IARNDGST (SEQ ID NO: 7) or IRKGNGST (SEQ ID NO: 8); and
(c) a heavy chain complementary determining region 3 (CDR_{H3}) having an amino acid sequence comprising ARMKKNGGSKRQPHIRS (SEQ ID NO: 16) or ATKAYIKSQLGS (SEQ ID NO: 17).

17. The single domain antibody of any one of claims 1 to 16, wherein the sdAb comprises:
(a) a heavy chain complementary determining region 1 (CDR_{H1}) having an amino acid sequence comprising GVKVSTKS (SEQ ID NO: 3), a heavy chain complementary determining region 2 (CDR_{H2}) having an amino acid sequence comprising IARNDGST (SEQ ID NO: 7) and a heavy chain complementary determining region 3 (CDR_{H3}) having an amino acid sequence comprising ARMKKNGGSKRQPHIRS (SEQ ID NO: 16); or
(b) a heavy chain complementary determining region 1 (CDR_{H1}) having an amino acid sequence comprising GFKVTSQA (SEQ ID NO: 4), a heavy chain complementary determining region 2 (CDR_{H2}) having an amino acid sequence comprising IRKGNGST (SEQ ID NO: 8) and a heavy chain complementary determining region 3 (CDR_{H3}) having an amino acid sequence comprising ATKAYIKSQLGS (SEQ ID NO: 17).

18. The single domain antibody of any one of claims 1 to 17, wherein the sdAb comprises an amino acid sequence having at least 75%, at least 80%, at least 85%, at least 90% or at least 95% sequence identity to SEQ ID NO: 18 or SEQ ID NO: 19.

19. The single domain antibody of any one of claims 1 to 18, wherein the sdAb comprises an amino acid sequence comprising SEQ ID NO: 18 or SEQ ID NO: 19.

20. An isolated nucleic acid encoding the single domain antibody of any one of claims 1 to 19.

21. The isolated nucleic acid of claim 20, having at least 85%, at least 90%, or at least 95% sequence identity to SEQ ID NO: 24 or SEQ ID NO: 25.

22. The isolated nucleic acid of claim 20 or 21 comprising SEQ ID NO: 24 or SEQ ID NO: 25.

23. An expression vector comprising a nucleic acid of any one of claims 20 to 22.

24. A host cell comprising the expression vector of claim 23.

25. A pharmaceutical composition comprising a single domain antibody as set forth in any one of claims 1 to 19 and a pharmaceutically acceptable carrier.

26. A method of treating a cancer in a subject in need thereof, the method comprising administering the pharmaceutical composition of claim 25 to the subject.

27. The method of claim 26 further comprising preventing, treating, or reducing a metastasis of the cancer in the subject.

28. The method of claim 26 or 27, wherein administering the single domain antibody disrupts formation of premetastatic niches in an organ in the subject.

29. The method of claim 28, wherein the organ is distal to a primary tumor in the subject.

30. The method of claim 28 or 29, wherein administering the single domain antibody disrupts rare heparin sulfate (rHS) signaling at a potential premetastatic niche in the subject.

31. The method of any one of claims 26 to 30 wherein the cancer comprises melanoma, lung cancer, breast cancer, colon cancer, kidney cancer, ovarian cancer, pancreatic cancer, prostate cancer, rectal cancer, stomach cancer, thyroid cancer, uterine cancer, brain tumors or a combination of any thereof.

32. The method of any one of claims 26 to 31, wherein the method further comprises administering a therapeutically effective amount of an anti-DC-HIL antibody to the subject.
